(19) United States
(12) Patent Application Publication
(10) Pub. No.: US 2016/0115188 A1
(43) Pub. Date: Apr. 28, 2016

(54) NOVEL alpha-GALACTOSYL CERAMIDE ANALOGS AND USES THEREOF

(71) Applicants: Chang Gung Memorial Hospital, Linkou, Taoyuan City (TW); National Chung Hsing Unviersity, Taichung (TW)

(72) Inventors: Shun-Yuan Luo, Taichung City (TW); Alice L. Yu, Kuei Shang, Taoyuan County (TW); Jung-Tung Hung, Kuei Shang, Taoyuan County (TW)

(21) Appl. No.: 14/848,614

(22) Filed: Sep. 9, 2015

Related U.S. Application Data
(60) Provisional application No. 62/069,790, filed on Oct. 28, 2014.

(51) Int. Cl. C07H 7/027

(52) U.S. Cl. CPC C07H 7/027

(57) ABSTRACT

The present invention is directed to compounds of formula (I) and pharmaceutical compositions comprising compounds of formula (I) and pharmaceutically acceptable carriers. The invention further comprises improved process for the preparation of compounds of formula (I), and the use of compound of formula (I) to induce a specific immune response or to treat an autoimmune disease.

### CROSS-REFERENCE TO RELATED APPLICATIONS

This application claims the benefit of U.S. Application No. 62/069,790, filed on 28 Oct. 2014, the entire disclosure of which is incorporated herein by reference.

### BACKGROUND OF THE INVENTION

The α-galactosyl ceramide (α-GalCer), also known as KRN7000, is a simplified glycolipid analogue of Agelasphins originally isolated from a marine sponge Agelas mauritianus.

α-GalCer binds to CD1d to form α-GalCer-CD1 complex, which is recognized by the T cell receptor of invariant natural killer T (iNKT) cells to generate a ternary complex. This recognition results in the rapid secretion of T helper Type 1 (Th1) and T helper Type 2 (Th2) cytokines but with only limited outcome in clinical trials. This is probably due to Th1 and Th2 cytokines antagonizing each other.

α-GalCer and its analogs play a critical role in vaccine adjuvants and modulating autoimmune disorders. There is still a need for more effective α-GalCer analogs in inducing Th1 or Th2 cytokine secretions and treating cancer and autoimmune diseases in a clinical setting, as well as a need for more efficient and economic ways to manufacture novel α-GalCer analogs. The present invention addresses these needs.

### SUMMARY OF THE INVENTION

The present invention relate to novel galactose-6-OH modified α-GalCer compounds (hereafter novel compound), pharmaceutical compositions comprising the novel compounds, methods of making the novel compounds, and methods comprising administering to a subject an effective amount of the novel compound.

In one embodiment, the present invention provides novel compounds having formula (I),

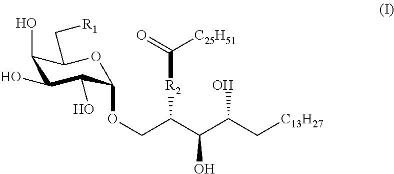

or a pharmaceutically acceptable salt thereof,

wherein R₁ is one of —O—R₃, —R₇N(R₈)R₉, —R₇₁N^{|}(R₇₂R₇₃R₇₄)X⁻, —R₁₁C(═NR₁₂)R₁₃, —R₁₄—N₃, —R₁₅—N═N—R₁₆, —R₁₇(C═O)R₁₈(C═O)R₁₉, or —R₂₀N(COR₂₁)(COR₂₂), where R₇ is a bond or alkenyl; each of R₈ and R₉ is independently a hydrogen, an alkyl or an alkenyl; R₇₁ is a bond or alkenyl; each of R₇₂, R₇₃ and R₇₄ is independently an alkyl or an alkenyl; X is a halogen; R₁₁ is a bond or an alkenyl; R₁₂ is an alkyl or alkenyl; R₁₃ is an alkyl or an alkenyl; R₁₄ is a bond or an alkenyl; R₁₅ is a bond or an alkenyl; R₁₆ is an alkyl or alkenyl; R₁₇ is a bond or alkenyl; R₁₈ is an alkenyl; R₁₉ is an alkyl or alkenyl; R₂₀ is a bond or an alkenyl; R₂₁ is an alkyl or an alkenyl; R₂₂ is an alkyl or an alkenyl;

R₂ is N—R₅ wherein R₅ is hydrogen or an alkyl;

R₃ is alkyl, alkenyl, —PO₃H₂, —SO₃Na, —SO₃K, —SO₃Li, —SH, —SR₆, —SSR₆, —SOR₆, —SO₂R₆, —SO₂H, —SO₃H, —SO₃R₆, —SCN, —R₆P, —OP(═O)(OH)₂ or —OPO(OR₆)₂, where R₆ is alkyl or an alkenyl;

provided R₁ is other than OCH₃ or NH₂ when R₂ is NH.

In another embodiment, the invention provides pharmaceutical compositions comprising a compound of formula (I) described herein and a pharmaceutical acceptable excipient or carrier.

A third embodiment of the present invention provides for the preparation methods of the novel compounds described herein.

In one exemplary embodiment, the present invention provides methods of preparing compounds having formula 2a

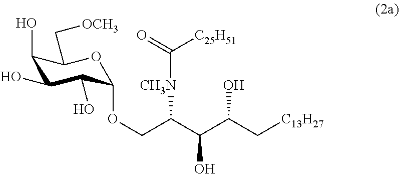

comprising:
(a) providing a compound having formula (9) (compound 9)

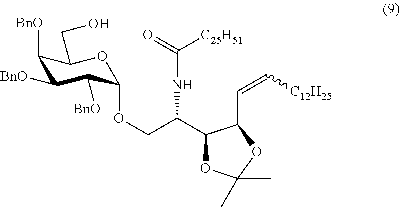

(b) reacting the compound (9) in (a) with

i) a base; and

ii) methyl iodide or DMS, to produce a compound having the formula (10a) (compound 10a)

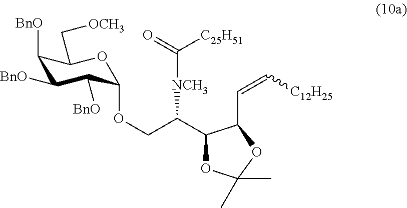

(c) converting the compound (10a) obtained in (b) to the compound having formula 2a.

In another exemplary embodiment, the present invention provides methods of preparing compounds having formula (II)

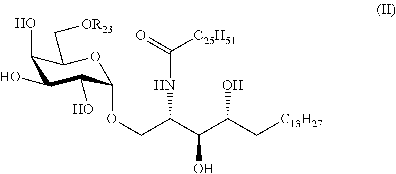

wherein R₂₃ is an alkyl having 1-30 carbon atoms or alkenyl having 2-30 carbon atoms, comprising:
(A) providing a compound having formula (9)

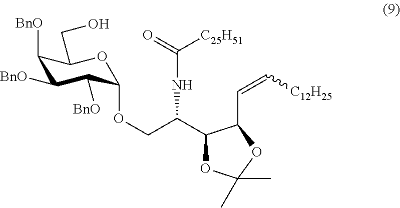

(B) reacting the compound (9) in (A) with

i) a base; and

ii) R₂₃ to produce a compound having formula (10)

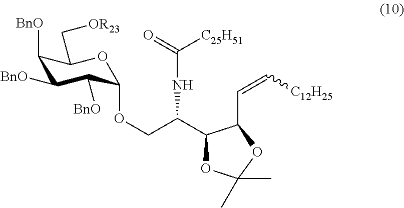

(C) converting the compound obtained in (B) to the compound having formula (II).

In one embodiment, reaction (B) is carried out in DMF at about 10° C. to about 35° C. or at about 4° C. to about −10° C. In another embodiment, the base in reaction (B) is NaH. In another embodiment, for the reaction in (b), for each of NaH and R₂₃I is added in an amount at a molar ratio of 2:1 of the amount of compound (9) obtained in (A), and R₂₃ is selected from CH₃, C₆H₁₃, C₁₂H₂₅, C₁₃H₂₇, and C₂₀H₄₁. In yet another embodiment, the reaction in (C) comprises reacting the compound obtained in (B) with a mixture containing palladium hydroxide, methanol, acetic acid, chloroform, and hydrogen gas.

In yet another exemplary embodiment, the present invention provides methods of preparing compounds having formula (2g)

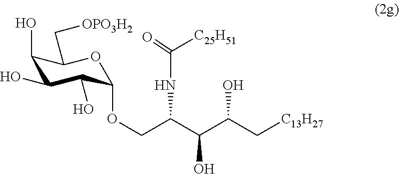

comprising:
(I) providing a compound having formula (9)

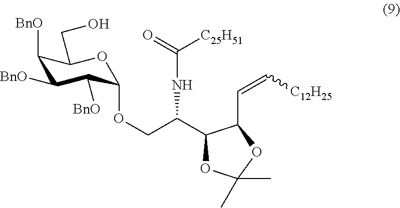

(II) reacting the compound (9) in (I) with diphenyl phosphoryl azide (DPPA) to produce a compound having formula (11g):

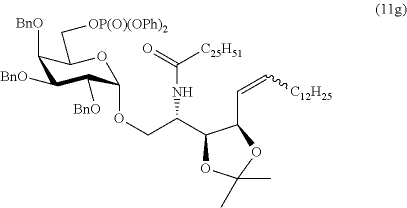

(III) hydrolyzing the acetonide group in the compound (11g) obtained from (b) to produce a diol compound having formula

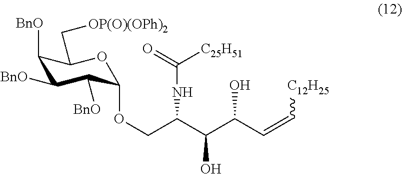

(IV) converting the compound (12) obtained in (III) to the compound having formula 2g.

In yet another exemplary embodiment, the present invention provides methods of preparing compounds having formula (2h):

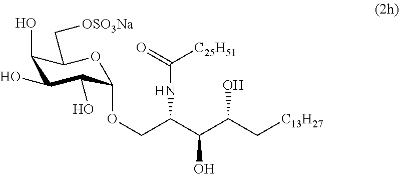

comprising:
(a) providing a compound having formula 8

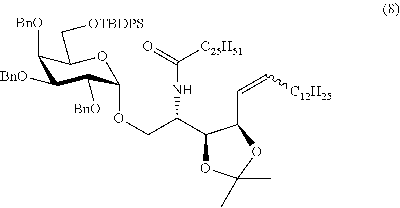

(b) hydrolyzing the acetonide group in the compound (8) obtained from (a) to produce a diol compound having the formula (14)

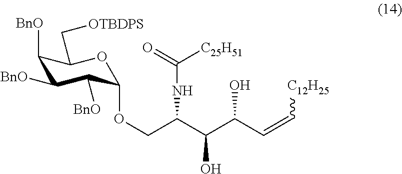

(c) benzylating the compound (14) produced in (b) to form a compound having the formula (15)

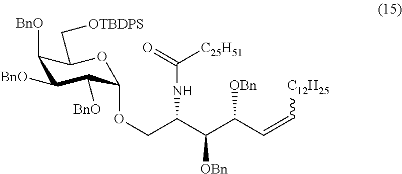

(d) hydrolyzing the compound (15) obtained in (c) to produce a compound having the formula (16)

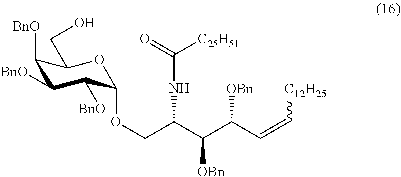

(e) converting the compound (16) obtained in (d) to the compound having formula (17)

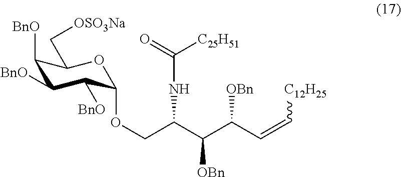

(f) converting the compound (17) obtained in (e) to the compound having the formula (2h).

In yet another exemplary embodiment, methods of preparing compounds having formula (2i)

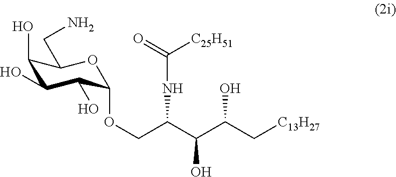

comprising:
(a) providing a compound having formula (9)

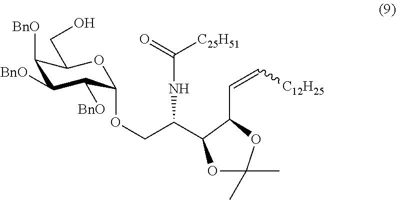

(b) converting the compound (9) in (a) to an azide compound having formula (18)

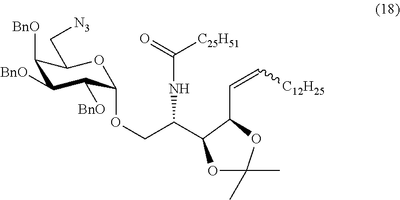

(c) converting the compound (18) obtained in (b) to form compound having the formula 2i.

A fourth embodiment of the present invention provides methods of increasing a serum cytokine, comprising the step of administering to a subject in need thereof an effective amount of a compound of formula (I) described herein or a pharmaceutically acceptable salt thereof.

The present invention is also directed to methods for (i) inducing or eliciting a Th2 immune response, comprises the step of administering to a subject in need thereof an effective amount of the compound of formula (I) described herein or a pharmaceutically acceptable salt thereof and (ii) inducing a Th1 immune response, comprising the step of administering to a subject in need thereof an effective amount of the compound of formula (I) described herein or a pharmaceutically acceptable salt thereof.

In an exemplary embodiment, Th2 immune response is induced by one or more of the following: Compound 2a, Compound 2b, Compound 2c, Compound 2d, Compound 2e, Compound 2f, Compound 2g, Compound 2h or Compound 2i. In another exemplary embodiment, Th1 immune response is induced by one or more of the following: Compound 2d, Compound 2e or Compound 2h.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

### BRIEF DESCRIPTION OF THE DRAWINGS

Illustrative embodiments of the present invention are described in detail below with reference to the following Figures:

FIG. 1 is a bar graph illustrating the induction of IL-2 by α-GalCer and compounds 2a-2i in mNK1.2 cells.

FIG. 2A is an assembly of bar graphs illustrating the cytokine levels of human iNKT cells co-cultured with dendritic cells loaded with α-GalCer or compounds 2a-2i.

FIG. 2B is an assembly of bar graphs illustrating the ratio of IL-4/IFN-γ and IL-10/IFN-γ of human iNKT cells co-cultured with dendritic cells loaded with α-GalCer or compounds 2a-2i.

### DETAILED DESCRIPTION OF THE INVENTION

In order to provide a clear and ready understanding of the present invention, certain terms are defined herein. Unless defined otherwise, all technical and scientific terms used herein have the same meanings as is commonly understood by one of skill in the art to which this invention belongs.

An “effective amount,” as used herein, refers to a dose of the compound of formula (I) or pharmaceutical composition that is sufficient to increase serum Th1 cytokine level or reduce the symptoms and signs of Th1 autoimmune disease, which include, but are not limited to, weight loss, skin rash, abdominal pain and joint pain.

The term “subject” can refer to a vertebrate having cancer or to a vertebrate deemed to be in need of autoimmune disease treatment. Subjects include warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

All numbers herein may be understood as modified by “about.”

### Compounds of Formula (I)

The present invention relates to a novel compound, which has the formula (I)

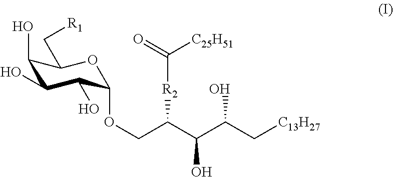

or a pharmaceutically acceptable salt thereof,

R₁ is one of —O—R₃, —R₇N(R₈)R₉, —R₇₁N⁺(R₇₂R₇₃R₇₄)X⁻, —R₁₁C(═NR₁₂)R₁₃, —R₁₄—N₃, —R₁₅—N═N—R₁₆, R₁₇(C═O)R₁₈(C═O)R₁₉, or —R₂₀N(COR₂₁)(COR₂₂), where R₇ is a bond or alkenyl; each of R₈ and R₉ is independently a hydrogen, an alkyl or an alkenyl; R₇₁ is a bond or alkenyl; each of R₇₂, R₇₃ and R₇₄ is independently an alkyl or an alkenyl; X is a halogen; R₁₁ is a bond or an alkenyl; R₁₂ is an alkyl or alkenyl; R₁₃ is an alkyl or an alkenyl; R₁₄ is a bond or an alkenyl; R₁₅ is a bond or an alkenyl; R₁₆ is an alkyl or alkenyl; R₁₇ is a bond or alkenyl; R₁₈ is an alkenyl; R₁₉ is an alkyl or alkenyl; R₂₀ is a bond or an alkenyl; R₂₁ is an alkyl or an alkenyl; R₂₂ is an alkyl or an alkenyl;

R₂ is N—R₅ where R₅ is hydrogen or an alkyl;

R₃ is alkyl, alkenyl, —PO₃H₂, —SO₃Na, —SO₃K, —SO₃Li, —SR₆, —SSR₆, —SOR₆, —SO₂R₆, —SO₂H, —SO₃R₆, —SCN, —R₆P, —OP(═O)(OH)₂ or —OPO(OR₆)₂, where R₆ is alkyl or an alkenyl;

provided R₁ is not OCH₃ or NH₂ when R₂ is NH.

Particular structures of compounds 2a-2i are listed in Table 1:

TABLE 1

| Compound | | R₁ | R₂ |
|---|---|---|---|
| 1. | (2S,3S,4R)-1-O-(6-O-methyl-α-D- | OCH₃ | NCH₃ |
| | galactopyranosyl)-D-ribo-2-N-methyl- | | |
| | hexacosanoylamino-1,3,4-octadecantriol | | |
| | (compound 2a) | | |
| 2. | (2S,3S,4R)-1-O-(6-O-methyl-α-D- | OCH₃ | NH |
| | galactopyranosyl)-D-ribo-2- | | |
| | hexacosanoylamino-1,3,4-octa-decantriol | | |
| | (compound 2b) | | |
| 3. | (2S,3S,4R)-1-O-(6-O-hexyl-α-D- | OC₆H₁₃ | NH |
| | galactopyranosyl)-D-ribo-2- | | |
| | hexacosanoylamino-1,3,4-octa-decantriol | | |
| | (compound 2c) | | |
| 4. | (2S,3S,4R)-1-O-(6-O-dodecyl-α-D- | OC₁₂H₂₅ | NH |
| | galactopyranosyl)-D-ribo-2- | | |
| | hexacosanoylamino-1,3,4-oct-adecantriol | | |
| | (compound 2d) | | |
| 5. | (2S,3S,4R)-1-O-(6-O-tridecyl-α-D- | OC₁₃H₂₇ | NH |
| | galactopyranosyl)-D-ribo-2- | | |
| | hexacosanoylamino-1,3,4-oct-adecantriol | | |
| | (compound 2e). | | |
| 6. | (2S,3S,4R)-1-O-(6-O-eicosanyl-α-D- | OC₂₀H₄₁ | NH |
| | galactopyranosyl)-D-ribo-2- | | |
| | hexacosanoylamino-1,3,4-oc-tadecantriol | | |
| | (compound 2f) | | |
| 7. | (2S,3S,4R)-1-O-(6-O-phospho-α-D- | OPO₃H₂ | NH |
| | galactopyranosyl)-D-ribo-2- | | |
| | hexacosanoylamino-1,3,4-oc-tadecantriol, | | |
| | phosphoric acid (compound 2g) | | |
| 8. | (2S,3S,4R)-1-O-(6-O-sulfo-α-D- | OSO₃Na | NH |
| | galactopyranosyl)-D-ribo-2- | | |
| | hexacosanoylamino-1,3,4-octade cantriol, | | |
| | sodium salt (compound 2h) | | |
| 9. | (2S,3S,4R)-1-O-(6-amine-α-D- | NH₂ | NH |
| | galactopyranosyl)-D-ribo-2- | | |
| | hexacosanoylamino-1,3,4-octadecantriol | | |
| | (compound 2i) | | |

“Alkyl” refers to groups of from 1 to 50 carbon atoms inclusively, either straight chained, branched, cyclic or unsaturated, preferably from 1 to 3 carbon atoms inclusively, from 1 to 4 carbon atoms inclusively, from 1 to 5 carbon atoms inclusively, from 1 to 30 carbon atoms inclusively, from 1 to 25 carbon atoms inclusively, preferably from 1 to 20 carbon atoms inclusively. Other chain lengths, e.g., 30-50, 30-40, 30-35, may be encompassed by the invention. Examples of alkyl include, but are not limited to, methyl, ethyl, n-propyl, i-propyl, n-butyl, i-butyl, and t-butyl.

“Alkynyl” refers to groups having 2 to 50 carbon atoms inclusively, either straight or branched containing at least one triple bond, preferably from 2 to 30 carbon atoms inclusively, more preferably from 2 to 20 carbon atoms inclusively. Other chain lengths, e.g., 30-50, 30-40, 30-35, may be encompassed by the invention.

“Halogen” represents a fluorine atom, a chlorine atom, a bromine atom, or an iodine atom.

“Bond,” as used herein, when a chemical group in a substituent is referred to as a bond, it is meant that the remaining part of the substituent is directly connected to the structure to be substituted via a single bond. For example, for compound A-B-C, where -B-C is a substituent for A, when B is referred to as a bond, it is understood that the compound will be A directly connected to C via a single bond, i.e, A-C.

Pharmaceutically acceptable salts of the compounds of formula (I) and physiologically functional derivatives thereof include salts derived from an appropriate base, such as an alkali metal (for example, sodium, potassium), an alkaline earth metal (for example, calcium, magnesium), ammonium and NY₄⁺ (wherein Y is C₁-C₄ alkyl). Pharmaceutically acceptable salts of an amino group include salts of organic carboxylic acids, such as tartaric, aliphatic, cycloaliphatic, aromatic, heterocyclic, carboxylic and sulfonic classes of organic acids, such as, for example, formic, glucuronic, malic, maleic, fumaric, pyruvic, aspartic, glutamic, benzoic, anthranilic, mesylic, salicylic, hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, hydroxybutyric, cyclochexylaminosulfonic, galactaric and galacturonic acid and the like, lactobionic, fumaric, and succinic acids; organic sulfonic acids, such as methaniesulfolic, ethanesulfonic, isothionic, benzenylesulfonic and p-toluenesulfonic acids; and inorganic acids such as hydrochloric, hydrobromic, hydroiodic, nitric, carbonic, sulfuric, sulfamic and phosphoric acid and the like. Pharmaceutically acceptable salts of a compound having a hydroxy group consist of the anion of said compound in combination with a suitable cation such as Na⁺, NH₄⁺ or NX₄⁺ (wherein X is, for example, a C₁-C₄ alkyl group), Ca⁺⁺, Li⁺⁺, Mg⁺⁺, or, K⁺ and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound in free form.

### The Pharmaceutical Composition

The present invention is also directed to pharmaceutical compositions comprising a compound of formula (I) or a pharmaceutically acceptable salt thereof, and a pharmaceutically acceptable carrier or excipient.

A “pharmaceutically acceptable carrier” refers to a carrier that, after administration to or upon a subject, does not cause undesirable physiological effects. The carrier in a pharmaceutical composition must be “acceptable” also in the sense that is compatible with the active compound and, preferably, capable of stabilizing it. Suitable pharmaceutically acceptable carriers are well known in the art and vary with the desired form and mode of administration of the pharmaceutical composition. For example, they may include, but are not limited to, biocompatible vehicles, adjuvants, additives (such as pH-adjusting additives), diluents or excipients such as fillers, binders, wetting agents, disintegrators, surface-active agents, lubricants and the like. The excipients may be nonionic surfactants, polyvinylpyrollidone, human serum albumin, aluminum hydroxide, agents with anesthetic action, and various unmodified and derivatized cyclodextrins. More preferably, the nonionic surfactants may include Polysorbate 20, Polysorbate 40, Polysorbate 60, and Polysorbate 80. The polyvinylpyrollidone may preferably be Plasdone C15, a pharmaceutical grade of polyvinylpyrollidone. The agent having anesthetic action preferably is benzyl alcohol. Other physiologically acceptable compounds include wetting agents, emulsifying agents, dispersing agents or preservatives. See e.g., the 21st edition of Remington's Pharmaceutical Science, Mack Publishing Company, Easton, Pa. (“Remington's”). The pharmaceutical compositions of the present invention can also include ancillary substances, such as pharmacological agents, cytokines, or other biological response modifiers. One or more pharmaceutical carriers may be used for the delivery of a compound of formula (I).

The pharmaceutical composition can be prepared by any method known in the art of pharmacy. Such methods include the step of bringing into association the active compound with one or more carriers. For instance, to prepare compositions suitable for injection, solutions and suspensions are sterilized and are preferably isotonic to blood. In making injectable preparations, carriers which are commonly used in this field are used, for example, water, ethyl alcohol, propylene glycol. In these instances, adequate amounts of isotonicity adjusters such as sodium chloride, glucose or glycerin can be added to make the preparations isotonic. The aqueous sterile injection solution may further comprise oxidants, buffers, and other similar additions, which are acceptable for parenteral compositions.

For instance, for oral administration in the form of a tablet or capsule, the active compound can be comminuted with a pharmaceutically acceptable carrier such as ethanol, glycerol, water and the like. Powders are prepared by comminuting the compound to a suitable fine size and mixing it with a comminuted pharmaceutical carrier such as an edible carbohydrate, for example, starch or mannitol. Flavoring, dispersing and coloring agents can also be present.

For the treatment of the eyes or other external tissues, for example, the mouth and the skin, the pharmaceutical compositions are preferably applied as a topical ointment or cream. When formulated in an ointment, the active compound may be employed with either a paraffinic or a water-miscible ointment base. Alternatively, the active compound may be formulated in a cream with an oil-in-water cream base or a water-in-oil base.

The processes of Producing the Compounds of Formula (I)

In one embodiment, compounds of formula (I) can be prepared using Compound 9 ((2S,3S,4R)-1-O-(2,3,4-tri-O-benzyl-α-D-galactopyranosyl)-2-hexacosanoylamino-3,4-O-iso-propylidene-5-octadecen-1,3,4-triol) as a starting material. Compound 9 can be prepared according to scheme 1.

The region- and stereoselective synthesis of Compound 5 (a disaccharide) can be prepared using the Gervay-Hague elegant glycosylation methodology, in which Compound 4 (galactosyl iodide) is generated in situ by treating 2,3,4-tri-O-benzyl-6-O-acetyl-α-D-galactopyranosyl acetate with iodotrimethylsilane. Compound 4 is added to Compound 3 (an acceptor) in the presence of TBAI and Hünig's base to provide Compound 5 as α-anomer.

Compound 6 (a disaccharide) is prepared by deacetylation of the O-6 position of the galactose moiety using sodium methoxide in methanol, followed by TBDPS protection, in two steps.

The Wittig olefination of hemiacetal 6 with C₁₃H₂₇PPh₃Br produced olefin Compound 7 in the presence of LiHMDS in tetrahydrofurane (THF) at about −10° C. to about 4° C. The successful azido displacement of alcohol 7 by using the Mitsunobu condition produced the desired azide compound.

Compound 8 (an amide product) is prepared by a subsequent Staudinger reaction, followed by amide bond formation in two steps.

The de-protection of the TBDPS group in the presence of 1 M of Tetra-n-butylammonium fluoride (TBAF) provided Compound 9 (a primary alcohol), the starting compound for preparing the compounds of formula (I).

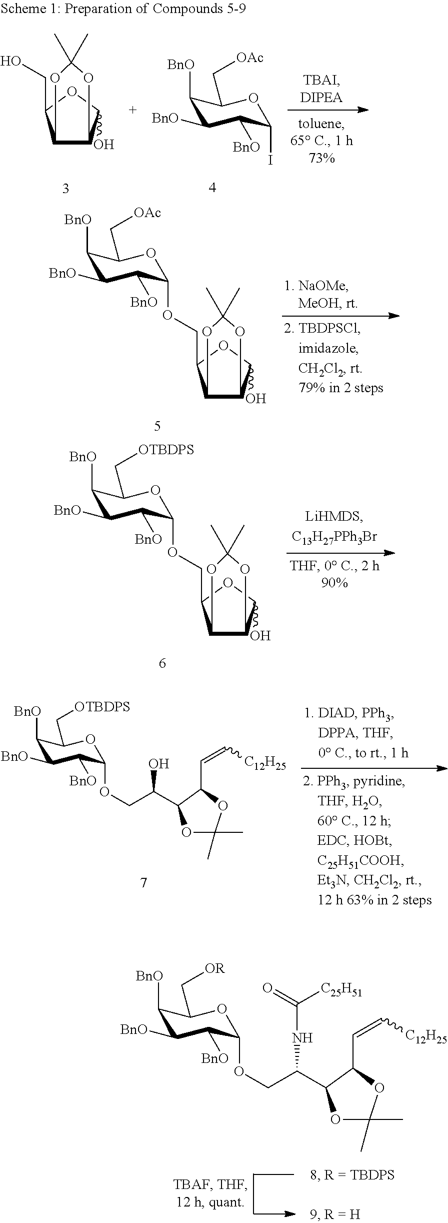

### Preparation of Compound 2a and Compound 2b

The preparation of compounds 2a and 2b begins with O- and N-methylation reaction of Compound 9 (Scheme 2).

In one embodiment, the reaction of Compound 9 with two equivalents of both a base (non-limiting example includes NaH) and methyl iodide or DMS in DMF at room temperature (for example, between 10° C. to 35° C., or between 15° C. to 30° C., or between 20° C. to 25° C.) produces a dimethylated product (Compound 10a) in about 12 h (Scheme 2 and Table 1, Route 1).

TABLE 1
Preparation of Compounds 2a and 2b

| | NaH | CH₃I | T | t | Yield | Yield |
|---|---|---|---|---|---|---|
| Route | (equiv) | (equiv.) | (° C.) | (h) | (10a) | (10b) |
| 1 | 2 | 2 | 25 | 12 | 81% | 0% |
| 2 | 2 | 2 | 25 | 8 | 76% | 21% |
| 3 | 2 | 2 | 0 | 2 | 0% | 64% |

In another embodiment, the reaction of Compound 9 with two equivalents of both a base (non-limiting example includes NaH) and methyl iodide or DMS in DMF at room temperature (for example, between 10° C. to 35° C., or between 15° C. to 30° C., or between 20° C. to 25° C.) produces a a mixture of compounds containing 76% of di-methylated Compound 10a and 21% of O-methylated Compound 10b in about 8 hours (Scheme 2 and Table 1, Route 2),

In another embodiment, the reaction of Compound 9 with two equivalents of both a base (non-limiting example includes NaH) and methyl iodide or DMS in DMF at 0° C. results in 64% yield of O-methylated Compound 10b in about 2 hours (Scheme 2 and Table 1, Entry 3).

In yet another embodiment, as illustrated in Scheme 2, Compounds 10a is mixed with a mixture comprising methanol, acetic acid, chloroform, and hydrogen gas to remove all of the benzyl groups and reduce the double bonds, and produces Compounds 2a (Scheme 2).

In yet another embodiment, as illustrated in Scheme 2, Compounds 10b is mixed with a mixture comprising methanol, acetic acid, chloroform, and hydrogen gas to remove the benzyl groups and reduce the double bonds, and produce Compounds 2b (Scheme 2).

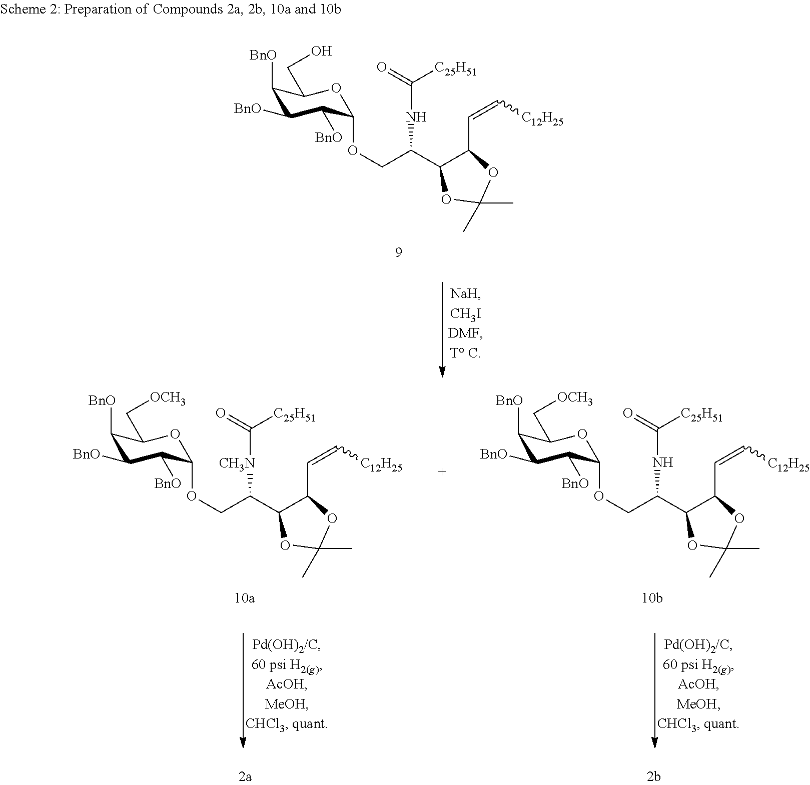

Preparation of Compound 2c-2g

In one embodiment, compound 9 is mixed with a base (non-limiting example includes NaH) and R₂₃I at 4° C. to −10° C. or room temperature to produce compounds having formula (10)

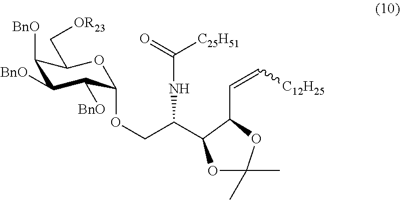

wherein R₂₃ is an alkyl having 1-30 carbon atoms or alkenyl having 2-30 carbon atoms.

In one embodiment, R₂₃ is C₆H₁₃ (Compound 11c), C₁₂H₂₅ (Compound 11d), C₁₃H₂₇ (Compound 11e) or C₂₀H₄₁ (Compound 11f), see Scheme 3.

In one embodiment, compound of formula (10) is deprotected to produce compounds having formula (II).

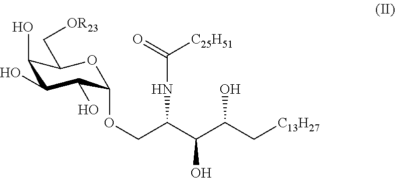

wherein R₂₃ is an alkyl having 1-30 carbon atoms or alkenyl having 2-30 carbon atoms.

In one embodiment, R₂₃ is C₆H₁₃ (Compound 2c), C₁₂H₂₅ (Compound 2d), C₁₃H₂₇ (Compound 2e) or C₂₀H₄₁ (Compound 2f), see Scheme 3.

In one embodiment, compound of formula (1) is mixed with a mixture comprising palladium hydroxide, methanol, acetic acid, chloroform, and hydrogen gas. In another embodiment, each of base and R₂₃ is added in an amount at a molar ratio of 2:1 of the amount of compound 9.

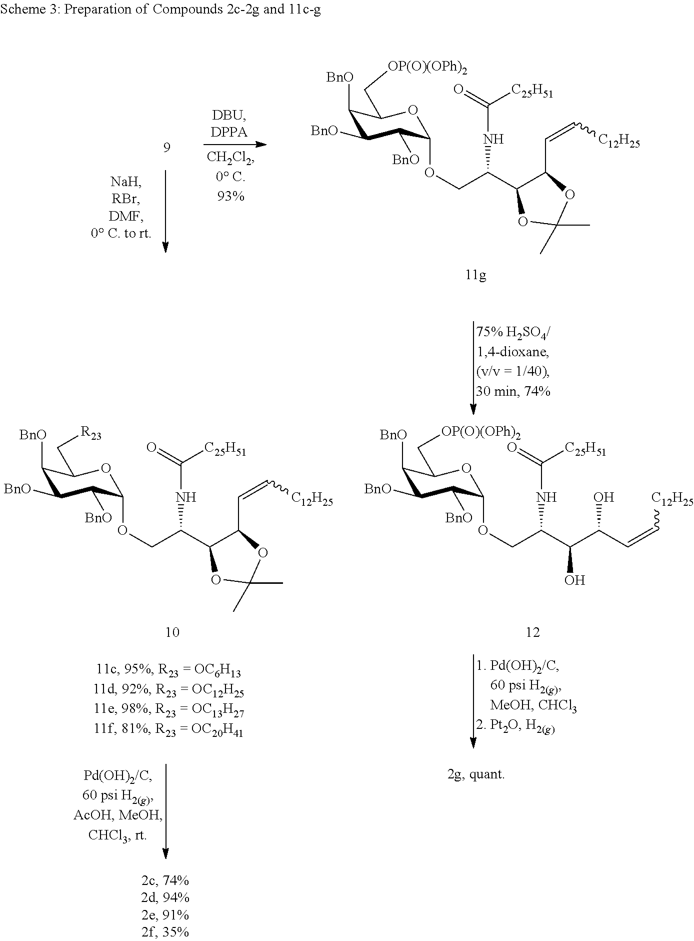

### Preparation of Compound 2g

In one embodiment, Compound 9 is reacted with DPPA to obtain the diphenylphosphoryl compound 11g in 93% yield (see Scheme 3). In one exemplary embodiment, the reaction of Compound 9 and DPPA is carried out at about 4° C. to −10° C. in the presence of a base in dichloromethane. Non limiting example of base includes 1,8-diazabicyclo[5.4.0]undec-7-ene.

In another embodiment, compound 11g undergoes direct global deprotection in an acidic condition to produce a diol compound 12 (see Scheme 3). In an exemplary embodiment, direct global deprotection is by hydrolyzing the acetonide group of compound 11g with an acid in ether. Non limiting example of acid is H₂SO₄, preferably 70%-80% of H₂SO₄, more preferably 75% of H₂SO₄. Non limiting example of ether is 1,4-dioxane.

In yet another embodiment, Compound 12 is converted to Compound 2g in the following reactions (see Scheme 3):
(i) reacting the compound 12 with a mixture containing palladium hydroxide, methanol, chloroform, and hydrogen gas, thereby obtaining a solution;
(ii) concentrating the solution in (i) to obtain a residue;
(iii) dissolving the residue in (ii) with a mixture of methanol and chloroform to obtain a second solution;
(iv)adding PtO₂ to the second solution; and
(v) passing hydrogen gas through the second solution.

### Preparation of Compound 2h

In one embodiment, Compound 8 is treated with an acid in the presence of ether, to cleave the acetonide group and produce a diol Compound 14 in a 64% yield (Scheme 4).

Non-limiting example of ether includes 1,4-dioxane and non-limiting examples of acid includes sulfuric acid

In another embodiment, benzylation of Compound 14 is carried out in the presence of NaH in THF to produce a fully protected compound 15 in a 68% yield (Scheme 4).

In yet another embodiment, the TBDPS group of Compound 15 was hydrolyzed using an ammonium salt, such as TBAF, in THF to yield a primary alcohol Compound 16.

In yet another embodiment, Compound 16 is treated with sulfur trioxide trimethylamine complex to generate Compound 17.

In yet another embodiment, Compound 17 is mixed with palladium hydroxide in a chloroform and methanol mixture with hydrogen gas to produce compound 2h.

Preparation of compound 2h by treating Compound 9 with sulfur trioxide in the presence of trimethylamine in DMF at 50° C. produces a sulfate compound 13. Deprotecting the benzyl groups of Compound 13 using palladium hydroxide to deprotect the benzyl groups was unsuccessful because the sensitivity of the sulfate and acetonide groups inhibited the deprotection of the benzyl groups. The use of strong acidic condition was subsequently led to the cleavage of glycosidic bond (See top portion of Scheme 4).

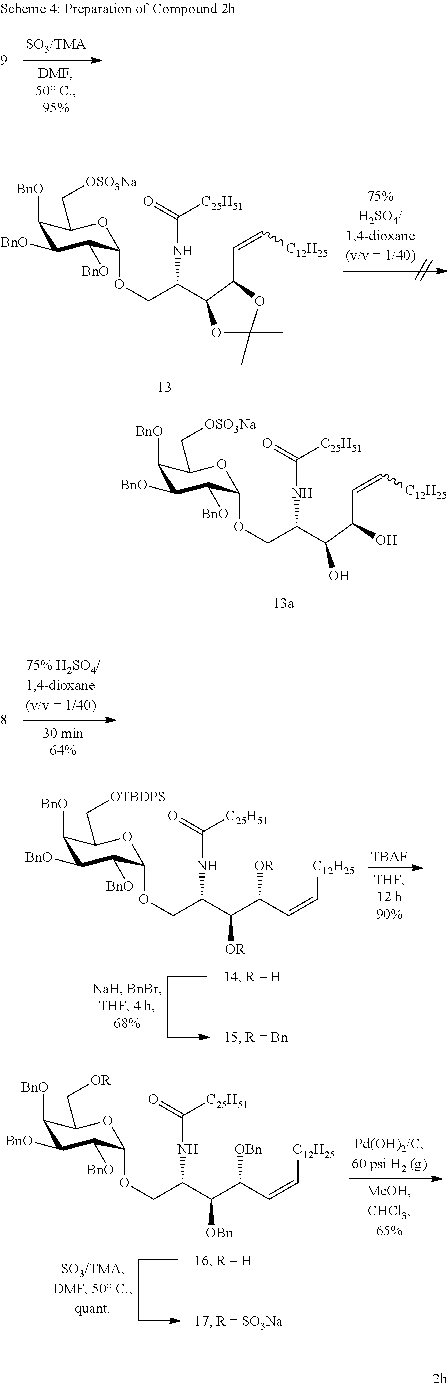

### Preparation of Compound 2i

In one embodiment, Compound 9 is mixed with (i) tetrahydrofurane containing triphenylphosphine to form a solution; followed by (ii) adding diisopropylazodicarboxylate and diphenylphosphorylazide to the solution in (i) to produce an azido Compound 18 (Scheme 5).

In another embodiment, compound 18 is deprotected to furnish an amine Compound 2i, by mixing Compound 18 with a mixture containing palladium hydroxide, methanol, acetic acid, chloroform, and hydrogen gas.

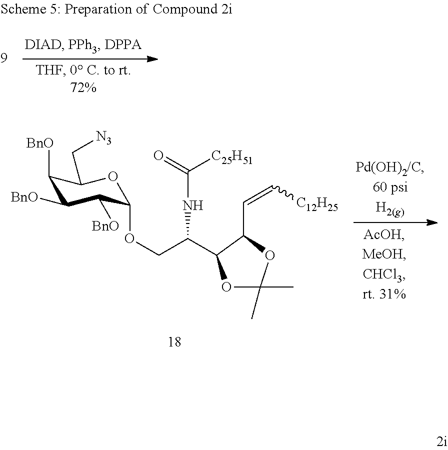

### Methods for Increasing a Cytokine in the Serum or Inducing Th1/Th2 Immune Response

Another aspect of the present invention is directed to methods for inducing or eliciting an immune response comprising administering an effective amount of the compound of formula (I)

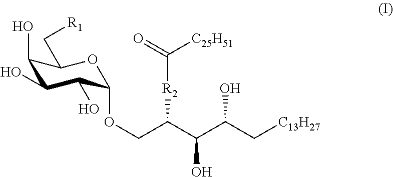

or a pharmaceutically acceptable salt thereof to a subject in need thereof,

R₁ is one of —O—R₃, —R₇N(R₈)R₉, —R₇₁N⁺(R₇₂R₇₃R₇₄)X⁻, —R₁₁C(═NR₁₂)R₁₃, —R₁₄—N₃, —R₁₅—N═N—R₁₆, or —R₁₇(C═O)R₁₈(C═O)R₁₉, —R₂₀N(COR₂₁)(COR₂₂), where R₇ is a bond or alkenyl; each of R₈ and R₉ is independently a hydrogen, an alkyl or an alkenyl; R₇₁ is a bond or alkenyl; each of R₇₂, R₇₃ and R₇₄ is independently an alkyl or an alkenyl; X is a halogen; R₁₁ is a bond or an alkenyl; R₁₂ is an alkyl or alkenyl; R₁₃ is an alkyl or an alkenyl; R₁₄ is a bond or an alkenyl; R₁₅ is a bond or an alkenyl; R₁₆ is an alkyl or alkenyl; R₁₇ is a bond or alkenyl; R₁₈ is an alkenyl; R₁₉ is an alkyl or alkenyl; R₂₀ is a bond or an alkenyl; R₂₁ is an alkyl or an alkenyl; R₂₂ is an alkyl or an alkenyl;

R₂ is N—R₅ where R₅ is hydrogen or an alkyl;

R₃ is alkyl, alkenyl, PO₃H₂, —SO₃Na, —SO₃K, SO₃Li, —SH, —SR₆, —SSR₆, —SOR₆, —SO₂R₆, —SO₂H, —SO₃H, —SO₃R₆, —SCN, —R₆P, —OP(═O)(OH)₂ or —OPO(OR₆)₂, where R₆ is alkyl or an alkenyl.

In one embodiment, the cytokine is a Th1 cytokine, selected from IFN-γ or IL-2. In another embodiment, the cytokine is a Th2 cytokine, selected from IL-4, IL-6 or IL-10. In another embodiment, the cytokine is GM-CSF. In yet another embodiment, R₃ is CH₃ to C₃₀H₆₁ or R₅ is CH₃ to C₆H₁₃. In yet another embodiment, R₃ is CH₂ and R₂ is NCH₃. In yet another embodiment, R₃ is CH₃—C₂₀H₄₁, PO₃H₂, SO₃Na or NH₂ and R₂ is NH.

The immune response includes but is not limited to, increasing a cytokine level in the serum of the subject, Th1 immune response or Th2 immune response.

In one exemplary embodiment, Th1 immune response is induced or elicited by administering one or more of the following compounds or the pharmaceutical composition thereof: Compound 2d, Compound 2e, or Compound 2h.

In another exemplary embodiment, Th2 immune response is induced or elicited by administering one or more of the following compounds or the pharmaceutical composition thereof: Compound 2a, Compound 2b, Compound 2c, Compound 2d, Compound 2e, Compound 2f, Compound 2g, Compound 2h or Compound 2i.

Another aspect of the present invention provides methods for treating Th1 dominated autoimmune disease, comprising administering an effective amount of the compound of formula (I)

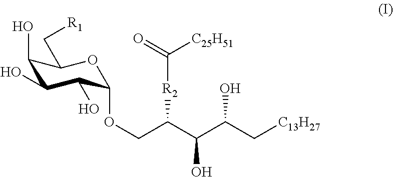

or the pharmaceutical composition thereof to a subject in need thereof

R₁ is one of —O—R₃, —R₇N(R₈)R₉, —R₇₁N⁺(R₇₂R₇₃R₇₄)X⁻, —R₁₁C(═NR₁₂)R₁₃, —R₁₄—N₃, —R₁₅—N═N—R₁₆, or —R₁₇(C═O)R₁₈(C═O)R₁₉, —R₂₀N(COR₂₁)(COR₂₂), where R₇ is a bond or alkenyl; each of R₈ and R₉ is independently a hydrogen, an alkyl or an alkenyl; R₇₁ is a bond or alkenyl; each of R₇₂, R₇₃ and R₇₄ is independently an alkyl or an alkenyl; X is a halogen; R₁₁ is a bond or an alkenyl; R₁₂ is an alkyl or alkenyl; R₁₃ is an alkyl or an alkenyl; R₁₄ is a bond or an alkenyl; R₁₅ is a bond or an alkenyl; R₁₆ is an alkyl or alkenyl; R₁₇ is a bond or alkenyl; R₁₈ is an alkenyl; R₁₉ is an alkyl or alkenyl; R₂₀ is a bond or an alkenyl; R₂₁ is an alkyl or an alkenyl; R₂₂ is an alkyl or an alkenyl;

R₂ is N—R₅ where R₅ is hydrogen or an alkyl

R₃ is alkyl, alkenyl, PO₃H₂, —SO₃Na, —SO₃K, SO₃Li, —SH, —SR₆, —SSR₆, —SOR₆, —SO₂R₆, —SO₂H, —SO₃H, —SO₃R₆, —SCN, —R₆P, —OP(═O)(OH)₂ or —OPO(OR₆)₂, where R6 is alkyl or an alkenyl.

In one embodiment, R₁ is OCH₃ and R₂ is NCH₃. In another embodiment, R₁ is OCH₃ to OC₂₀H₄₁ and R₂ is NH. In yet another embodiment, R₁ is OSO₃Na or OPO₃H₂ and R₂ is NH. In yet another embodiment, R₁ is NH₂ and R₂ is NH.

Th1 dominated disease is treated by eliciting the Th2 immune response. In one embodiment, Th2 immune response is characterized by increasing the secretion of Th2 cytokines such as IL-4, IL-6 or IL-10. In another embodiment, Th2 immune response is characterized by increasing the ratio of IL-4/IFN-γ and IL-10/IFN-γ. Non-limiting examples of Th1 autoimmune disease include Type I diabetes, multiple sclerosis, Hashimoto's Thyroiditis, Grave's disease, Crohn's disease, psoriasis, Sjoren's Syndrome, celiac disease, lichen planus and rheumatoid arthritis.

Another aspect of the present invention provides methods for treating Th2 dominated autoimmune disease, comprising administering an effective amount of the compound of formula (I)

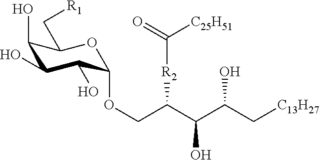

or the pharmaceutical composition thereof to a subject in need thereof,

R₁ is one of —O—R₃, —R₇N(R₈)R₉, —R₇₁N⁺(R₇₂R₇₃R₇₄)X⁻, —R₁₁C(═NR₁₂)R₁₃, —R₁₄—N₃, —R₁₅—N═N—R₁₆, or —R₁₇(C═O)R₁₈(C═O)R₁₉, —R₂₀N(COR₂₁)(COR₂₂), where R₇ is a bond or alkenyl; each of R₈ and R₉ is independently a hydrogen, an alkyl or an alkenyl; R₇₁ is a bond or alkenyl; each of R₇₂, R₇₃ and R₇₄ is independently an alkyl or an alkenyl; X is a halogen; R₁₁ is a bond or an alkenyl; R₁₂ is an alkyl or alkenyl; R₁₃ is an alkyl or an alkenyl; R₁₄ is a bond or an alkenyl; R₁₅ is a bond or an alkenyl; R₁₆ is an alkyl or alkenyl; R₁₇ is a bond or alkenyl; R₁₈ is an alkenyl; R₁₉ is an alkyl or alkenyl; R₂₀ is a bond or an alkenyl; R₂₁ is an alkyl or an alkenyl; R₂₂ is an alkyl or an alkenyl;

R₂ is N—R₅ where R₅ is hydrogen or an alkyl

R₃ is alkyl, alkenyl, PO₃H₂, —SO₃Na, —SO₃K, SO₃Li, —SH, —SR₆, —SSR₆, —SOR₆, —SO₂R₆, —SO₂H, —SO₃H, —SO₃R₆, —SCN, —R₆P, —OP(═O)(OH)₂ or —OPO(OR₆)₂, where R6 is alkyl or an alkenyl.

In one embodiment, R₁ is OC₁₂H₁₅ to OC₁₃H₂₇and R₂ is NH. In another embodiment, R₁ is OSO₃Na and R₂ is NH.

Th2 dominated disease is treated by eliciting the Th1 immune response. In one embodiment, Th1 immune response is characterized by increasing the secretion of Th1 cytokines such as IFN-γ or IL-2. In another embodiment, Th1 immune response is characterized by inducing T cell proliferation.

Non-limiting examples of Th2 autoimmune disease include lupus allergic dermatitis, scleroderma, atopic eczema, sinusitis, inflammatory bowel disease, asthma, and ulcerative colitis.

The term “administering” covers inhalation, topical, oral, rectal, implanted reservoir and parenteral (such as intravenous, intramuscular, subcutaneous, intra-articular, intra-synovial, cisternal, intrathecal, intrahepatic, intralesional and intracranial) delivery to a subject the active compound of the invention. Parenteral route of administration is preferred.

The composition for oral administration can be any orally acceptable dosage form including capsules, tablets, emulsions and aqueous suspensions, dispersions, and solutions. The oral composition may include sustained release properties as well as rapid delivery forms.

Topical application may be formulated in carriers such as hydrophobic or hydrophilic bases to form ointments, cream, lotions, in aqueous, oleaginous or alcoholic liquids to form paints or in dry diluents to form powders.

The parenteral compositions may take such forms as suspensions, solutions, or emulsions in oily or aqueous vehicles, and may contain various formulating agents. Alternatively, the active ingredient may be in powder form for reconstitution at the time of delivery with a suitable vehicle, such as sterile water.

The compounds of formula (I) described herein or the pharmaceutical compositions thereof can be administered in a single dose treatment or in multiple dose treatments on a schedule and over a time period appropriate to the age, weight and condition of the subject, the particular composition used, and the route of administration, whether the pharmaceutical composition is used for prophylactic or curative purposes, etc. For example, in one embodiment, the pharmaceutical composition according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of the compounds of formula (I) described herein or the pharmaceutical compositions thereof, e.g., the period of time over which the compound or the pharmaceutical composition is administered, can vary, depending on any of a variety of factors, e.g., subject response, etc. For example, the compound or the pharmaceutical composition can be administered over a period of time ranging from about one or more seconds to one or more hours, one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

For ease of administration and uniformity of dosage, oral or parenteral pharmaceutical compositions in dosage unit form may be used. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The data obtained from the cell culture assays and animal studies can be used in formulating a range of dosage for use in humans. In one embodiment, the dosage of such compounds lies within a range of circulating concentrations that include the ED₅₀ with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Sonderstrup, Springer, Sem. Immunopathol. 25: 35-45, 2003. Nikula et al., Inhal. Toxicol. 4(12): 123-53, 2000.

The following examples further illustrate the present invention. These examples are intended merely to be illustrative of the present invention and are not to be construed as being limiting.

### EXAMPLES

Material and Methods: Dichloromethane, tetrahydrofuran, toluene, methanol, and N,N-dimethyformamide were purified and dried from a safe purification system containing activated Al₂O₃. All reagents obtained from commercial sources were used without purification, unless otherwise specified. Flash column chromatography was carried out on Silica Gel 60 (230-400 mesh). TLC was performed on pre-coated glass plates of Silica Gel 60 F254 (0.25 mm); detection was executed by spraying with a solution of Ce(NH₄)₂(NO₃)₆ (0.5 g), (NH₄)₆Mo₇O₂₄ (24 g) and H₂SO₄ (28 mL) in water (500 mL) and subsequent heating on a hot plate. Optical rotations were measured at 589 nm (Na) at ˜27° C. ¹H, ¹³C NMR, DEPT, ¹H-¹H COSY, ¹H-¹³C COSY, and ¹H-¹H NOESY spectra were recorded with 400 and 600 MHz instruments. Chemical shifts are in ppm from Me₄Si, generated from the CDCl₃ lock signal at δ 7.24 ppm. IR spectra were taken with a FT-IR spectrometer using KBr plates. Mass spectra were analyzed on an Orbitrap instrument with an ESI source.

### Example 1

### Preparation of Compound 5 (5-O-(6-O-acetyl-2,3,4-tri-O-benzyl-α-D-galactopyranosyl)-2,3-O-isopropylidene-D-lyxofura-nose)

To a solution of 6-O-acetyl-2,3,4-tri-O-benzyl-α-D-galactopyranosyl acetate (8.23 g, 15.4 mmol) in dichloromethane (80 mL) was added iodotrimethylsilane (TMSI, 2.74 mL, 19.3 mmol) at 0° C. under nitrogen. After stirring for 30 min, the reaction was stopped by adding anhydrous toluene. The mixture was azeotroped with toluene three times. The iodide Compound 4 was dissolved in toluene and kept under N₂. A mixture of 2,3-O-isopropylidene-D-lyxofuranose 3 (3.22 g, 16.9 mmol), diisopropylethylamine (DIPEA, 2.68 mL, 15.4 mmol), tetrabutyl ammonium iodide (TBAI, 17.1 g, 46.2 mmol) and 4 Å molecular sieves (4.00 g) was added into anhydrous toluene (50 mL) and was stirred for 10 min at 65° C. under nitrogen. Then a solution of Compound 4 in toluene was cannulated into the reaction flask, the mixture was kept stirring for 1 h at 65° C., and the reaction was stopped by adding ethyl acetate. The reaction mixture as cooled to 0° C., the white precipitate and molecular sieves was removed by filtration through celite. The filtrate was extracted with aqueous Na₂S₂O₃ (80 mL) and brine, and the organic layers were dried over anhydrous MgSO₄, filtered, and concentrated in vacuo. The residue was purified by column chromatography on silica gel to afford the desired disaccharide Compound 5 (7.50 g) as colorless oil in 73% yield over two steps. R_{f} 0.47 (EtOAc/Hex=1/1); [α]²⁴ _{D} +3.92 (c 1.2, CHCl₃); IR (CHCl₃) v 3404, 2925, 1742 cm⁻¹; ¹H NMR (600 MHz, CDCl₃) δ 7.41-7.26 (m, 15H, ArH), 5.38 (bs, 1H, H-1), 4.97 (d, J=11.4 Hz, 1H, PhCH₂), 4.87 (d, J=11.4 Hz, 1H, PhCH₂), 4.86 (d, J=3.0 Hz, 1H, H-1′), 4.82 (d, J=12.0 Hz, 1H, PhCH₂), 4.75 (d, J=11.4 Hz, 1H, PhCH₂), 4.75-4.73 (m, 1H, H-3), 4.68 (d, J=12.0 Hz, 1H, PhCH₂), 4.62 (d, J=11.4 Hz, 1H, PhCH₂), 4.57 (d, J=6.0 Hz, 1H, H-2), 4.39-4.37 (m, 1H, H-4), 4.24-4.21 (m, 1H, H-6a′), 4.06-3.96 (m, 4H, H-2′, H-3′, H-5′, H-6b′), 3.90-3.86 (m, 2H, H-5a, H-4′), 3.78 (dd, J=11.4, 4.8 Hz, 1H, H-5b), 3.30 (bs, 1H, OH), 1.98 (s, 3H, CH₃), 1.42 (m, 3H, CH₃), 1.28 (s, 1H, CH₃); ¹³C NMR (150 MHz, CDCl₃) δ 171.0 (C), 138.7 (C), 138.4 (C), 138.2 (C), 128.4 (CH×2), 128.33 (CH×2), 128.31 (CH×2), 128.3 (CH×2), 127.9 (CH×2), 127.69 (CH), 127.68 (CH), 127.5 (CH), 127.4 (CH×2), 112.4 (C), 101.0 (CH), 98.1 (CH), 85.4 (CH), 80.0 (CH), 79.0 (CH), 78.9 (CH), 76.5 (CH), 74.6 (CH), 74.5 (CH₂), 73.4 (CH₂), 73.3 (CH₂), 68.0 (CH), 67.1 (CH₂), 63.2 (CH₂), 26.0 (CH₃), 24.7 (CH₃), 20.1 (CH₃); HRMS (ESI, M+Na⁺) calculated for C₃₇H₄₄O₁₁Na 687.2776, found 687.2779.

### Example 2

### Preparation of Compound 6 (5-O-(2,3,4-tri-O-benzyl-6-O-tert-butyldiphenylsilyl-α-D-gala-ctopyranosyl)-2,3-O-isopropyli-dene-D-lyxofuranose)

To a solution of compound 5 (2.15 g, 3.24 mmol) and sodium methoxide (70 mg, 1.30 mmol) in methanol (25 mL) was stirred for 4 h and concentrated in vacuo. After the crude disaccharide was dissolved in dichloromethane (20 mL), imidazole (0.66 g, 9.71 mmol) and tert-butylchlorodiphenylsilane (0.9 mL, 3.40 mmol) were added to the solution, and the mixture was continuously stirred for 2 h. The reaction solution was washed by water (20 mL). The organic layer was dried over anhydrous MgSO₄, filtered, and concentrated in vacuo. Purification of this residue via column chromatography gave the disaccharide Compound 6 (2.20 g, 79% in 2 steps) as colorless oil. R_{f} 0.28 (EtOAc/Hex=1/3); [α]²⁴_{D} +5.70 (c 1.0, CHCl₃); IR (CHCl₃) v 3406, 2932, 2857 cm⁻¹; ¹H NMR (600 MHz, CDCl₃) δ 7.72-7.29 (m, 25H, ArH), 5.50 (d, J=1.8 Hz, 1H, H-1), 5.07 (d, J=11.4 Hz, 1H, CH₂Ph), 4.96 (d, J=11.4 Hz, 1H, CH₂Ph), 4.93 (d, J=3.0 Hz, 1H, H-1′), 4.88 (d, J=12.0 Hz, 1H, CH₂Ph), 4.83 (d, J=12.0 Hz, 1H, CH₂Ph), 4.79-4.78 (m, 1H, H-3), 4.77 (d, J=11.7 Hz, 1H, CH₂Ph), 4.69 (d, J=11.7 Hz, 1H, CH₂Ph), 4.63 (d, J=6.0 Hz, 1H, H-2), 4.50-4.48 (m, 1H, H-4), 4.14-4.09 (m, 3H, H-2′, H-3′, H-4′), 3.88 (m, 1H, H-5′), 3.90-3.75 (m, 4H, H-5a, H-5b, H-6a′, H-6b′), 3.68 (bs, 1H, OH), 1.45 (s, 3H, CH₃), 1.34 (s, 3H, CH₃), 1.15 (s, 9H, CH₃); ¹³C NMR (150 MHz, CDCl₃) δ 138.9 (C), 138.7 (C), 138.5 (C), 135.4 (CH×4), 133.20 (C), 133.18 (C), 129.61 (CH), 129.59 (CH), 128.24 (CH×2), 128.18 (CH×2), 128.0 (CH×2), 127.9 (CH×2), 127.8 (CH×2), 127.7 (CH×2), 127.6 (CH×2), 127.5 (CH), 127.33 (CH), 127.29 (CH×3), 112.3 (C), 100.9 (CH), 97.8 (CH), 85.3 (CH), 79.8 (CH), 78.8 (CH), 78.4 (CH), 76.4 (CH), 75.1 (CH), 74.8 (CH₂), 72.95 (CH₂), 72.92 (CH₂), 70.5 (CH), 65.9 (CH₂), 62.2 (CH₂), 26.8 (CH₃×3), 26.0 (CH₃), 24.7 (CH₃), 19.1 (C); HRMS (APCI, M+Na⁺) calculated for C₅₁H₆₀O₁₀NaSi 883.3848, found 883.3857.

### Example 3

### Preparation of Compound 7 (2R,3S,4R)-1-O-(2,3,4-tri-O-benzyl-6-O-tert-butyldiphenylsi-lyl-α-D-galactopyranosyl)-3,4-O-isopropylidene-5-octadecen-1,2,3,4-tetraol)

A mixture of hemiacetal Compound 6 (2.77 g, 3.21 mmol) and tridecanyltriphenylphosphonium bromide (6.76 g, 12.9 mmol) in tetrahydrofuran (27 mL) was cooled to 0° C. under nitrogen. A 1.0 M solution of lithium hexamethyldisilamide in tetrahydrofuran (LiHMDS, 12.9 mL, 12.9 mmol) was added to the reaction mixture and stirred for another 2 h at 0° C. Water (30 mL) was added to quench the reaction and the mixture was extracted with ethyl acetate (2×30 mL). The combined organic layers were washed with brine, dried over anhydrous MgSO₄, filtered, and concentrated in vacuo to give a residue. The residue was purified by column chromatography to give the olefin Compound 7 (2.93 g, 89%) as colorless oil. R_{f} 0.61 (EtOAc/Hex=1/3); [α]²⁴_{D} +3.36 (c 0.9, CHCl₃); IR (CHCl₃) v 2926, 2855, 1456, 1104 cm⁻¹; ¹H NMR (400 MHz, CDCl₃) δ 7.62-7.20 (m, 25H, ArH), 5.74-5.63 (m, 2H, H-5, H-6), 4.95 (d, J=11.6 Hz, 1H, CH₂Ph), 4.96-4.92 (m, 1H, H-4), 4.86 (d, J=12.0 Hz, 1H, CH₂Ph), 4.80 (d, J=12.0 Hz, 1H, CH₂Ph), 4.77 (d, J=3.6 Hz, 1H, H-1′), 4.75 (d, J=11.6 Hz, 1H, CH₂Ph), 4.67 (d, J=12.0 Hz, 1H, CH₂Ph), 4.58 (d, J=11.2 Hz, 1H, CH₂Ph), 4.13-4.09 (m, 1H, H-3), 4.03-4.00 (m, 2H, H-2′, H-3′), 3.94 (dd, J=10.4, 2.8 Hz, 1H, H-4′), 3.88 (t, J=2.8 Hz, 1H, H-5′), 3.78-3.65 (m, 3H, H-2, H-6a′, H-6b′), 3.56 (dd, J=10.4, 7.2 Hz, 1H, H-1a), 3.58 (dd, J=10.8, 7.2 Hz, 1H, H-1b), 2.58 (d, J=6.4, 1H, OH), 2.14-1.93 (m, 2H, CH₂), 1.49 (s, 3H, CH₃), 1.36 (s, 3H, CH₃), 1.36-1.33 (m, 2H, CH₂), 1.28-1.24 (m, 18H, CH₂), 1.04 (s, 9H, CH₃), 0.88 (t, J=6.4 Hz, 3H, CH₃); ¹³C NMR (150 MHz, CDCl₃) δ 138.8 (C), 138.7 (C), 138.5 (C), 135.5 (CH×5), 133.22 (C), 133.21 (C), 129.69 (CH), 129.67 (CH), 128.32 (CH×2), 128.30 (CH×2), 128.1 (CH×2), 128.0 (CH×2), 127.9 (CH×2), 127.7 (CH×4), 127.6 (CH), 127.5 (CH), 127.38 (CH), 127.37 (CH×2), 125.0 (CH), 108.4 (C), 97.7 (CH), 79.0 (CH), 77.3 (CH), 76.4 (CH), 74.9 (CH), 74.8 (CH₂), 73.3 (CH₂), 72.99 (CH₂), 72.97 (CH), 70.9 (CH), 69.6 (CH₂), 68.4 (CH), 62.4 (CH₂), 31.9 (CH₂), 29.7 (CH₂), 29.64 (CH₂), 29.61 (CH₂), 29.57 (CH₂), 29.49 (CH₂), 29.46 (CH₂), 29.3 (CH₂), 29.2 (CH₂), 27.7 (CH₂), 27.2 (CH₃), 26.9 (CH₃×3), 24.9 (CH₃), 22.7 (CH₂), 19.1 (C), 14.1 (CH₃); HRMS (ESI, M+Na⁺) calculated for C₆₄H₈₆O₉NaSi 1049.5933, found 1049.5954.

### Example 4

### Preparation of Compound 8 ((2S,3S,4R)-1-O-(2,3,4-tri-O-benzyl-6-O-tert-butyldiphenyl-silyl-α-D-galactopyranosyl)-2-hexacosanoylamino-3,4-O-iso-propylidene-5-octadecen-1,3,4-triol)

To a solution of Compound 7 (396 mg, 0.39 mmol) and triphenylphosphine (307 mg, 1.16 mmol) in tetrahydrofuran (4.0 mL) at 0° C. was added diisopropyl azodicarboxylate (DIAD, 235 μL, 1.16 mmol), followed by dropwise addition of DPPA (269 μL, 1.25 mmol). After completion of addition, the reaction was brought to room temperature and stirred for 1 h. Water (5 mL) was added to quench the reaction and the mixture was extracted with ethyl acetate (2×5 mL). The combined organic layers were washed with brine, dried over anhydrous MgSO₄, filtered, and concentrated in vacuo to give a residue. The residue was purified by column chromatography to give the azide (405 mg, quant.) as colorless oil. To a solution of azide (405 mg, 0.38 mmol) and triphenylphosphine (202 mg, 0.77 mmol) in THF (4.0 mL) was added pyridine (1.3 mL). The reaction flask was warmed up to 60° C., and the mixture was kept stirring for 12 h. The reaction was gradually cooled to room temperature, hexaeicosanoic acid (199 mg, 0.50 mmol), 1-[3-(dimethylamino)propyl]-3-ethylcarbodiimide hydrochloride (EDC, 133 mg, 0.69 mmol), hydroxybenzotriazole (HOBt, 94 mg, 0.69 mmol) and triethylamine (54 μL, 0.39 mmol) were sequentially added to the solution, and the mixture was continuously stirred for 12 h. The reaction solution was diluted with ethyl acetate (3.0 mL), and the resulting mixture was washed by water (8.0 mL). The organic layer was dried over anhydrous MgSO₄, filtered, and concentrated in vacuo. Purification of this residue via column chromatography gave the amide Compound 8 (337 mg, 63%) as colorless oil. R_{f} 0.46 (EtOAc/Hex=1/5); [α]²⁴_{D} +5.20 (c 1.0, CHCl₃); IR (CHCl₃) v 2923, 2853, 1680, 1537 cm⁻¹; ¹H NMR (600 MHz, CDCl₃) δ 7.61-7.23 (m, 25H, ArH), 5.98 (d, J=9.0 Hz, 1H, NH), 5.59-5.54 (m, 1H, H-6), 5.43-5.40 (m, 1H, H-5), 5.02 (d, J=3.6 Hz, 1H, H-1′), 4.96 (d, J=10.8 Hz, 1H, CH₂Ph), 4.83 (d, J=10.8 Hz, 1H, CH₂Ph), 4.83-4.81 (m, 1H, H-4), 4.80 (d, J=11.4 Hz, 1H, CH₂Ph), 4.78 (d, J=10.8 Hz, 1H, CH₂Ph), 4.68 (d, J=10.8 Hz, 1H, CH₂Ph), 4.59 (d, J=11.4 Hz, 1H, CH₂Ph), 4.16 (dd, J=9.0, 6.0 Hz, 1H, H-3), 4.11-4.09 (m, 1H, H-2), 4.07 (d, J=3.0 Hz, 1H, H-4′), 4.05 (dd, J=10.2, 3.6 Hz, 1H, H-2′), 3.92 (dd, J=10.2, 3.0 Hz, 1H, H-3′), 3.80-3.77 (m, 2H, H-5′, H-6a′), 3.75 (dd, J=11.4, 3.0 Hz, 1H, H-1a), 3.68 (dd, J=13.2, 9.0 Hz, 1H, H-6b′), 3.58 (dd, J=11.4, 3.0 Hz, 1H, H-1b), 2.07-1.86 (m, 6H, CH₂), 1.55-1.53 (m, 2H, CH₂), 1.42 (s, 3H, CH₃), 1.35 (s, 3H, CH₃), 1.33-1.24 (m, 62H, CH₂), 1.05 (s, 9H, CH₃), 0.88 (t, J=7.2 Hz, 6H, CH₃×2); ¹³C NMR (150 MHz, CDCl₃) δ 172.1 (C), 138.7 (C), 138.6 (C), 138.3 (C), 135.5 (CH×4), 135.0 (CH), 133.2 (C), 133.1 (C), 129.8 (CH), 129.7 (CH), 128.4 (CH×4), 128.1 (CH×2), 127.94 (CH×2), 127.90 (CH×2), 127.8 (CH), 127.74 (CH×2), 127.71 (CH×2), 127.6 (CH), 127.43 (CH×2), 127.41 (CH), 124.1 (CH), 108.3 (C), 98.2 (CH), 78.9 (CH), 76.9 (CH), 76.0 (CH), 74.9 (CH₂), 74.6 (CH), 73.5 (CH₂), 73.1 (CH), 72.6 (CH₂), 70.9 (CH), 67.5 (CH₂), 62.2 (CH₂), 48.7 (CH), 36.8 (CH₂), 31.9 (CH₂×2), 29.7 (CH₂×19), 29.60 (CH₂×2), 29.56 (CH₂×3), 29.5 (CH₂×2), 29.4 (CH₂×3), 27.9 (CH₃), 27.7 (CH₂), 26.9 (CH₃×3), 25.7 (CH₃), 25.5 (CH₂), 22.7 (CH₂), 19.1 (C), 14.1 (CH₃×2); HRMS (ESI, M+H⁺) calculated for C₉₀H₁₃₈O₉NSi 1405.0135, found 1405.0104.

### Example 5

### Preparation of Compound 9 ((2S,3S,4R)-1-O-(2,3,4-tri-O-benzyl-α-D-galactopyranosyl)-2-hexacosanoylamino-3,4-O-iso-propylidene-5-octadecen-1,3,4-triol)

To a solution of the Compound 8 (194 mg, 0.14 mmol) in tetrahydrofuran (2.0 mL) was added 1.0 M solution of tetrabutylammonium fluoride in tetrahydrofuran (TBAF, 280 μL, 0.28 mmol) and stirred for 12 h. Water (3 mL) was added to quench the reaction and the mixture was extracted with ethyl acetate (2×3 mL). The combined organic layers were washed with brine, dried over anhydrous MgSO₄, filtered, and concentrated in vacuo to give a residue. The residue was purified by column chromatography to afford Compound 9 (161 mg, quant.) as white solid. R_{f} 0.19 (EtOAc/Hex=1/3); [α]²⁵_{D} +8.83 (c 0.6, CHCl₃); mp 66° C.; IR (CHCl₃) v 3424, 2918, 2850, 1644 cm⁻¹; ¹H NMR (600 MHz, CDCl₃) δ 7.41-7.26 (m, 15H, ArH), 5.98 (d, J=9.0 Hz, 1H, NH), 5.64-5.60 (m, 1H, H-6), 5.46-5.43 (m, 1H, H-5), 4.98 (d, J=3.6 Hz, 1H, H-1′), 4.96 (d, J=12.0 Hz, 1H, CH₂Ph), 4.91-4.89 (m, 1H, H-4), 4.82 (d, J=11.4 Hz, 2H, CH₂Ph), 4.76 (d, J=11.4 Hz, 1H, CH₂Ph), 4.70 (d, J=11.4 Hz, 1H, CH₂Ph), 4.64 (d, J=12.0 Hz, 1H, CH₂Ph), 4.19-4.13 (m, 2H, H-2, H-3), 4.06 (dd, J=10.2, 3.6 Hz, 1H, H-2′), 3.94-3.87 (m, 3H, H-1a, H-3′, H-4′), 3.73-3.65 (m, 3H, H-1b, H-5′, H-6a′), 3.54-3.52 (m, 1H, H-6b′), 2.21 (s, 1H, OH), 2.10-1.93 (m, 4H, CH₂), 1.54-1.53 (m, 2H, CH₂), 1.46 (s, 3H, CH₃), 1.36 (s, 3H, CH₃), 1.33-1.24 (m, 64H, CH₂), 0.88 (t, J=7.2 Hz, 6H, CH₃×2); ¹³C NMR (150 MHz, CDCl₃) δ 172.6 (C), 138.5 (C), 138.2 (C), 138.1 (C), 135.6 (CH), 128.5 (CH×2), 128.4 (CH×6), 128.0 (CH×2), 127.89 (CH), 127.85 (CH), 127.6 (CH), 127.4 (CH×2), 123.8 (CH), 108.3 (C), 99.4 (CH), 79.1 (CH), 77.0 (CH), 76.7 (CH), 74.6 (CH), 74.6 (CH₂), 73.5 (CH₂), 73.1 (CH), 73.0 (CH₂), 70.9 (CH), 69.1 (CH₂), 62.2 (CH₂), 49.5 (CH), 36.8 (CH₂), 31.9 (CH₂×2), 29.7 (CH₂×22), 29.50 (CH₂), 29.46 (CH₂), 29.42 (CH₂), 29.38 (CH₂), 29.3 (CH₂×2), 27.8 (CH₂), 27.4 (CH₃), 25.5 (CH₂), 25.4 (CH₃), 22.7 (CH₂×2), 14.1 (CH₃×2); HRMS (ESI, M+H⁺) calculated for C₇₄H₁₂₀O₉N 1166.8958, found 1166.8931.

### Example 6

### Preparation of Compound 10a ((2S,3S,4R)-1-O-(2,3,4-tri-O-benzyl-6-O-methyl-α-D-galacto-pyranosyl)-2-N-methyl-hexaco-sanoylamino-3,4-O-isopropyli-dene-5-octadecen-1,3,4-triol)

To a solution of Compound 9 (32 mg, 0.03 mmol) in N,N-dimethylformamide (DMF, 1 mL) were added iodomethane (4 μL, 0.06 mmol) and 60% sodium hydride (22 mg, 0.06 mmol) at 28° C. After complete addition, the reaction mixture was stirred for 2 h. Methanol was added to quench the reaction and concentrated in vacuo. The mixture was extracted with ethyl acetate (3×5 mL) and water (5 mL). The combined organic layers were dried over anhydrous MgSO₄, filtered, and concentrated in vacuo to give a residue. The residue was purified by column chromatography to produce Compound 10a (27 mg, 81%) as a yellow solid. R_{f} 0.50 (EtOAc/Hex=1/2.5); [α]²⁵_{D} +14.3 (c1.0, CHCl₃); IR (CHCl₃) v 2924, 2853, 1651, 1370, 1057 cm⁻¹; ¹H NMR (600 MHz, C₅D₅N, 100° C.) δ 7.53-7.27 (m, 15H, ArH), 5.84 (t, J=10.8 Hz, 1H, H-5), 5.79 (bs, 1H, H-6), 5.22 (bs, 2H, H-4, H-1′), 5.16 (d, J=11.4 Hz, 1H, PhCH₂), 4.98 (d, J=11.4 Hz, 1H, PhCH₂), 4.92 (d, J=11.4 Hz, 1H, PhCH₂), 4.84-4.79 (m, 3H, PhCH₂), 4.36 (dd, J=11.4, 3.0 Hz, 1H, H-2′), 4.25-4.23 (m, 4H, H-2, H-3′, H-5′, H-6a′), 4.10 (m, 1H, H-6b′), 3.84-3.80 (m, 3H, H-1a, H-3, H-4′), 3.71 (t, J=6.6 Hz, 1H, H-1b), 3.39 (s, 3H, CH₃), 3.13 (s, 3H, CH₃), 2.39-2.25 (m, 3H, CH₂), 1.86 (bs, 1H, CH₂), 1.58 (s, 3H, CH₃), 1.53-1.49 (m, 4H, CH₂), 1.45 (s, 3H, CH₃), 1.40 (bs, 62H, CH₂), 0.932 (t, J=6.0 Hz, 3H, CH₃), 0.928 (t, J=6.0 Hz, 3H, CH₃); ¹³C NMR (150 MHz, C₅D₅N, 100° C.) δ 173.7 (C), 140.1 (C), 140.0 (C×2), 135.1 (CH), 128.83 (CH×2), 128.76 (CH×3), 128.71 (CH×2), 128.5 (CH×2), 128.2 (CH×3), 127.90 (CH), 127.86 (CH×2), 126.6 (CH), 108.7 (C), 99.0 (CH), 79.8 (CH), 78.0 (CH), 77.8 (CH), 77.0 (CH), 75.5 (CH₂), 74.4 (CH×2), 73.3 (CH₂), 72.4 (CH₂), 70.6 (CH), 67.5 (CH₂), 59.1 (CH₃), 34.5 (CH₂), 33.3 (CH₃), 32.3 (CH₂×2), 30.1 (CH₂×21), 30.0 (CH₂×2), 29.73 (CH₂×2), 29.69 (CH₂×2), 29.66 (CH₂×2), 28.22 (CH₂), 28.18 (CH₃), 25.80 (CH₃), 25.73 (CH₂), 23.0 (CH₂×2), 14.2 (CH₃×2); HRMS (ESI, M+H³⁰ ) calculated for C₇₆H₁₂₄O₉N 1194.9271, found 1194.9259.

### Example 7

### Preparation of Compound 10b ((2S,3S,4R)-1-O-(2,3,4-tri-O-benzyl-6-O-methyl-α-D-galacto-pyranosyl)-2-hexacosanoylamino-3,4-O-isopropylidene-5-oc-tadecen-1,3,4-triol)

To a solution of Compound 9 (17 mg, 0.01 mmol) in N,N-dimethylformamide (DMF, 1.0 mL) were added iodomethane (2 μL, 0.03 mmol) and 60% sodium hydride (1 mg, 0.03 mmol) at 0° C. After complete addition, the reaction mixture was stirred for 2 h. Methanol was added to quench the reaction and concentrated in vacuo. The mixture was extracted with ethyl acetate (3×5 mL) and water (5 mL). The combined organic layers were dried over anhydrous MgSO₄, filtered, and concentrated in vacuo to give a residue. The residue was purified by column chromatography to afford Compound 10b (11 mg, 64%) as a yellow solid. R_{f} 0.38 (EtOAc/Hex=1/2.5); [α]²⁵_{D} +21.9 (c 0.9, CHCl₃); mp 59-59.6° C.; IR (CHCl₃) v 3314, 2918, 2850, 1643, 1469, 1054 cm⁻¹; ¹H NMR (600 MHz, CDCl₃) δ 7.34-7.20 (m, 15H, ArH), 6.26 (d, J=9.6 Hz, 1H, NH), 5.51 (td, J=10.8, 7.2 Hz, 1H, H-6), 5.35 (t, J=10.2 Hz, 1H, H-5), 4.88 (d, J=11.4 Hz, 1H, PhCH₂), 4.83 (d, J=3.6 Hz, 1H, H-1′), 4.78 (dd, J=9.6, 5.4 Hz, 1H, H-4), 4.75 (d, J=12.0 Hz, 1H, PhCH₂), 4.73 (d, J=12.0 Hz, 1H, PhCH₂), 4.68 (d, J=12.0 Hz, 1H, PhCH₂), 4.60 (d, J=12.0 Hz, 1H, PhCH₂), 4.55 (d, J=11.4 Hz, 1H, PhCH₂), 4.13 (dd, J=9.0, 5.4 Hz, 1H, H-3), 4.01-3.96 (m, 3H, H-2, H-1a, H-2′), 3.84-3.83 (m, 3H, H-3′, H-4′, H-5′), 3.55 (dd, J=9.6, 2.4 Hz, 1H, H-1b), 3.38 (dd, J=9.6, 7.2 Hz, 1H, H-6a′), 3.21-3.18 (m, 4H, H-6b′, OCH₃), 2.05-1.81 (m, 4H, CH₂), 1.49-1.41 (m, 2H, CH₂), 1.38 (s, 3H, CH₃), 1.28 (s, 3H, CH₃), 1.18 (bs, 64H, CH₂), 0.81 (t, J=6.6 Hz, 6H, CH₃×2); ¹³C NMR (150 MHz, CDCl₃) δ 172.4 (C), 138.6 (C), 138.3 (C), 138.3 (C),134.8 (CH), 128.4 (CH×6), 128.3 (CH×2), 127.9 (CH×2), 127.8 (CH), 127.7 (CH), 127.6 (CH), 127.5 (CH×2), 124.2 (CH), 108.3 (C), 99.6 (CH), 78.8 (CH), 76.7 (CH), 75.8 (CH), 74.61 (CH₂), 74.60 (CH), 73.4 (CH₂), 72.98 (CH), 72.95 (CH₂), 72.0 (CH₂), 70.5 (CH₂), 69.7 (CH), 58.9 (CH₃), 49.0 (CH), 36.6 (CH₂), 31.9 (CH₂), 29.7 (CH₂×12), 29.64 (CH₂×5), 29.58 (CH₂×3), 29.52 (CH₂×2), 29.46 (CH₂×3), 29.37 (CH₂×2), 29.3 (CH₂×3), 28.0 (CH₃), 27.6 (CH₂), 25.7 (CH₃), 25.4 (CH₂), 22.7 (CH₂), 14.1 (CH₃×2); HRMS (ESI, M+Na⁺) calculated for C₇₅H₁₂₁O₉NNa 1202.8934, found 1202.8933.

### Example 8

### Preparation of Compound 2a ((2S,3S,4R)-1-O-(6-O-methyl-α-D-galactopyranosyl)-D-ribo-2-N-methyl-hexacosanoylamino-1,3,4-octadecantriol)

Compound 10a (17 mg) was dissolved in a mixed solvent of MeOH/CHCl₃ (3/1 ratio, 2 mL) at 28° C. The Pd(OH)₂/C (17 mg, Degussa type) was added to the solution and followed by addition 2-3 drops of acetic acid, the reaction vessel was purged with hydrogen, and the mixture was stirred under 60 psi pressure at the same temperature for 5 h. The resulting solution was filtered through celite, the filtrate was concentrated in vacuo, and the residue was purified by column chromatography to afford Compound 2a (12 mg, quant.) as white solid. R_{f} 0.13 (MeOH/DCM=1/10); [α]²⁵_{D} +46.3 (c 0.1, CHCl₃); mp 64-66° C.; IR (CHCl₃) v 3324, 2920, 2851, 1652, 1036 cm⁻¹; ¹H NMR (600 MHz, d-pyridine, 100° C.) δ 5.24 (d, J=4.2 Hz, 1H, H-1′), 4.60 (dd, J=10.8, 4.2 Hz, 1H, H-1a), 4.39 (dd, J=9.6, 3.6 Hz, 1H, H-2′), 4.35 (t, J=6.0 Hz, 1H, H-5′), 4.31 (bs, 1H, H-4′), 4.27-4.25 (m, 2H, H-1b, H-3′), 4.21 (bs, 1H, H-3), 4.15 (dd, J=6.0, 1.8 Hz, 1H, H-2), 4.05-4.03 (m, 1H, H-4), 3.97 (dd, J=10.2, 5.4 Hz, 1H, H-6a′), 3.88 (dd, J=9.6, 6.0 Hz, 1H, H-6b′), 3.40 (s, 3H, CH₃), 3.27 (s, 3H, CH₃), 2.50-2.36 (m, 1H, CH₂), 2.10-2.08 (m, 1H, CH₂), 1.87-1.80 (m, 4H, CH₂), 1.68-1.62 (m, 1H, CH₂), 1.52-1.44 (m, 6H, CH₂), 1.39 (bs, 30H, CH₂), 1.35 (bs, 31H, CH₂), 0.93 (t, J=13.2 Hz, 6H, CH₃×2); ¹³C NMR (150 MHz, d-pyridine, 100° C.) δ 174.6 (C), 101.4 (CH), 76.8 (CH), 73.6 (CH), 73.2 (CH₂), 71.7 (CH), 71.0 (CH×2), 70.6 (CH), 67.7 (CH, CH₂), 59.1 (CH₃), 35.0 (CH₃), 34.5 (CH₂), 34.0 (CH₂), 32.2 (CH₂×2), 30.4 (CH₂), 30.1 (CH₂×26), 29.7 (CH₂×2), 26.6 (CH₂), 25.7 (CH₂), 23.0 (CH₂×2), 14.2 (CH₃×2); HRMS (ESI, M+H⁺) calculated for C₅₂H₁₀₄O₉N 886.77056, found 886.77062.

### Example 9

### Preparation of Compound 2b ((2S,3S,4R)-1-O-(6-O-methyl-α-D-galactopyranosyl)-D-ribo-2-hexacosanoylamino-1,3,4-octa-decantriol

Compound 10b (22 mg) was dissolved in a mixed solvent of MeOH/CHCl₃ (3/1 ratio, 2 mL) at 28° C. The Pd(OH)₂/C (22 mg, Degussa type) was added to the solution and followed by addition 2-3 drops of acetic acid, the reaction vessel was purged with hydrogen, and the mixture was stirred under 60 psi pressure at the same temperature for 5 h. The resulting solution was filtered through celite, the filtrate was concentrated in vacuo, and the residue was purified by column chromatography to afford Compound 2b (16 mg, quant.) as white solid. R_{f} 0.31 (MeOH/DCM=1/10); [α]²⁵_{D} +25.0 (c 0.04, CHCl₃); mp 86-88° C.; IR (CHCl₃) v 3274, 2918, 2850, 1641 cm⁻¹; ¹H NMR (600 MHz, d-pyridine) δ 8.47 (d, J=9.0 Hz, 1H, NH), 6.48 (bs, 1H, OH), 5.52 (d, J=3.6 Hz, 1H, H-1′), 5.27-5.23 (m, 1H, H-2), 4.64 (dd, J=10.8, 5.4 Hz, 1H, H-1a), 4.61 (dd, J=10.2, 4.2 Hz, 1H, H-2′), 4.46 (t, J=6.0 Hz, 1H, H-5′), 4.40-4.36 (m, 3H, H-1b, H-3′, H-4′), 4.34-4.30 (m, 2H, H-3, H-4), 3.97 (dd, J=9.6, 5.4 Hz, 1H, H-6a′), 3.94 (dd, J=10.2, 6.6 Hz, 1H, H-6b′), 3.33 (s, 3H, CH₃), 2.43 (m, 2H, CH₂), 2.30-2.25 (m, 1H, CH₂), 1.95-1.86 (m, 2H, CH₂), 1.84-1.78 (m, 2H, CH₂), 1.71-1.62 (m, 2H, CH₂), 1.30 (bs, 26H, CH₂), 1.23 (bs, 39H, CH₂), 0.850 (t, J=7.2 Hz, 3H, CH₃), 0.847 (t, J=6.6 Hz, 3H, CH₃); ¹³C NMR (150 MHz, d-pyridine) δ 173.1 (C), 101.5 (CH), 76.5 (CH), 73.0 (CH₂), 72.5 (CH), 71.3 (CH), 70.8 (CH), 70.7 (CH), 70.1 (CH), 68.8 (CH₂), 58.7 (CH₃), 51.2 (CH), 36.8 (CH₂), 34.2 (CH₂), 32.1 (CH₂×2), 30.3 (CH₂), 30.1 (CH₂), 30.0 (CH₂×20), 29.92 (CH₂×3), 29.86 (CH₂×2), 29.82 (CH₂), 29.75 (CH₂), 29.6 (CH₂×2), 26.5 (CH₂), 26.4 (CH₂), 22.9 (CH₃×2); HRMS (ESI, M+H⁺) calculated for C₅₁H₁₀₂O₉N 872.7549, found 872.7536.

### Example 10

### Preparation of Compound 11c ((2S,3S,4R)-1-O-(2,3,4-tri-O-benzyl-6-O-hexyl-α-D-galacto-pyranosyl)-2-hexacosanoylamino-3,4-O-isopropylidene-5-oc-tadecen-1,3,4-triol)

To a solution of the alcohol 9 (33 mg, 0.03 mmol) in N,N-dimethylformamide (1 mL) were added 1-bromohexane (8 μL, 0.06 mmol) and 60% sodium hydride (2 mg, 0.06 mmol) at 28° C. After complete addition, the reaction mixture was stirred for 8 h. Methanol was added to quench the reaction and concentrated in vacuo. The mixture was extracted with ethyl acetate (3×5 mL) and water (5 mL). The combined organic layers were dried over anhydrous MgSO₄, filtered, and concentrated in vacuo to give a residue. The residue was purified by column chromatography to afford Compound 11c (34 mg, 95%) as a yellow solid. R_{f} 0.64 (EtOAc/Hex=1/2.5); [α]²⁵_{D} +26.3 (c 0.6, CHCl₃); mp 43-44° C.; IR (CHCl₃) v 3317, 2920, 2851, 1646, 1537, 1468, 1055 cm⁻¹; ¹H NMR (600 MHz, CDCl₃) δ 7.34-7.19 (m, 15H, ArH), 6.17 (d, J=9.6 Hz, 1H, NH), 5.51 (td, J=10.8, 7.2 Hz, 1H, H-6), 5.35 (t, J=9.6 Hz, 1H, H-5), 4.87 (d, J=11.4 Hz, 1H, PhCH₂), 4.87 (d, J=3.6 Hz, 1H, H-1′), 4.78 (dd, J=9.6, 6.0 Hz, 1H, H-4), 4.73 (d, J=10.8 Hz, 2H, PhCH₂), 4.68 (d, J=12.0 Hz, 1H, PhCH₂), 4.61 (d, J=11.4 Hz, 1H, PhCH₂), 4.55 (d, J=12.0 Hz, 1H, PhCH₂), 4.14 (dd, J=9.0, 6.0 Hz, 1H, H-3), 4.02-3.96 (m, 2H, H-2, H-2′), 3.91 (dd, J=11.4, 3.0 Hz, 1H, H-1a), 3.87 (bs, 1H, H-4′), 3.85-3.81 (m, 2H, H-3′, H-5′), 3.56 (dd, J=10.8, 2.4 Hz, 1H, H-1b), 3.38 (dd, J=9.0, 6.0 Hz, 1H, H-6a′), 3.33 (td, J=10.2, 6.6 Hz, 1H, CH₂), 3.29 (dd, J=9.0, 6.0 Hz, 1H, H-6b′), 3.22 (td, J=9.6, 7.2 Hz, 1H, CH₂), 2.00 (dddd, J=15.0, 7.2, 7.2, 7.2 Hz, 1H, CH₂), 1.97-1.88 (m, 2H, CH₂), 1.38 (s, 3H, CH₃), 1.28 (s, 3H, CH₃), 1.18 (bs, 70H, CH₂), 0.82 (t, J=6.6 Hz, 3H, CH₃), 0.81 (t, J=7.2 Hz, 6H, CH₃×2); ¹³C NMR (150 MHz, CDCl₃) δ 172.4 (C), 138.6 (C), 138.4 (C), 138.3 (C), 135.0 (CH), 128.4 (CH×2), 128.34 (CH×2), 128.30 (CH×2), 128.2 (CH×2), 127.9 (CH×2), 127.8 (CH), 127.7 (CH), 127.54 (CH), 127.46 (CH×2), 124.1 (CH), 108.3 (C), 99.2 (CH), 78.8 (CH), 76.7 (CH), 75.8 (CH), 74.7 (CH₂), 74.5 (CH), 73.4 (CH₂), 73.0 (CH), 72.8 (CH₂), 71.6 (CH₂), 69.57 (CH₂), 69.56 (CH), 69.45 (CH₂), 49.0 (CH), 36.7 (CH₂), 34.7 (CH₂), 31.9 (CH₂×2), 29.72 (CH₂×5), 29.68 (CH₂×8), 29.64 (CH₂×3), 29.59 (CH₂), 29.55 (CH₂), 29.48 (CH₂×2), 29.4 (CH₂×2), 29.3 (CH₂×2), 28.0 (CH₃), 27.7 (CH₂), 25.71 (CH₂), 25.68 (CH₃), 25.4 (CH₂), 22.7 (CH₂×2), 22.6 (CH₂), 14.1 (CH₃×2), 14.0 (CH₃); HRMS (ESI, M+H⁺) calculated for C₈₀H₁₃₂O₉N 1250.98966, found 1250.98974.

### Example 11

### Preparation of Compound 11d ((2S,3S,4R)-1-O-(2,3,4-tri-O-benzyl-6-O-dodecyl-α-D-gala-ctopyranosyl)-2-hexacosanoylamino-3,4-O-isopropylidene-5-octadecen-1,3,4-triol)

To a solution of Compound 9 (33 mg, 0.03 mmol) in N,N-dimethylformamide (1 mL) were added 1-bromododecane (14 μL, 0.06 mmol) and 60% sodium hydride (2 mg, 0.06 mmol) at 28° C. After complete addition, the reaction mixture was stirred for 8 h. Methanol was added to quench the reaction and concentrated in vacuo. The mixture was extracted with ethyl acetate (3×5 mL) and water (5 mL). The combined organic layers were dried over anhydrous MgSO₄, filtered, and concentrated in vacuo to give a residue. The residue was purified by column chromatography to afford Compound 11d (35 mg, 93%) as a yellow solid. R_{f} 0.64 (EtOAc/Hex=1/2.5); [α]²⁵_{D} +28.5 (c 0.4, CHCl₃); mp 49-50° C.; IR (CHCl₃) v 3353, 2918, 2860, 1662, 1531, 1468, 1043 cm⁻¹; ¹H NMR (600 MHz, CDCl₃) δ 7.41-7.28 (m, 15H, ArH), 6.16 (d, J=8.4 Hz, 1H, NH), 5.58 (td, J=10.8, 7.2 Hz, 1H, H-6), 5.42 (d, J=10.2 Hz, 1H, H-5), 4.94 (d, J=11.4 Hz, 1H, PhCH₂), 4.92 (d, J=3.6 Hz, 1H, H-1′), 4.85 (dd, J=9.0, 6.0 Hz, 1H, H-4), 4.81 (d, J=12.0 Hz, 1H, PhCH₂), 4.80 (d, J=10.8 Hz, 1H, PhCH₂), 4.75 (d, J=11.4 Hz, 1H, PhCH₂), 4.68 (d, J=11.4 Hz, 1H, PhCH₂), 4.62 (d, J=11.4 Hz, 1H, PhCH₂), 4.21 (dd, J=9.6, 6.0 Hz, 1H, H-3), 4.07 (td, J=9.0, 3.0 Hz, 1H, H-2), 4.05 (dd, J=9.6, 3.0 Hz, 1H, H-2′), 4.50 (dd, J=11.4, 3.0Hz, 1H, H-1a), 3.94 (bs, 1H, H-4′), 3.92-3.89 (m, 2H, H-3′, H-5′), 3.64 (dd, J=11.4, 2.4 Hz, 1H, H-1b), 3.46 (dd, J=9.6, 6.6 Hz, 1H, H-6a′), 3.40 (dt, J=9.0, 6.6 Hz, 1H, CH₂), 3.35 (dd, J=9.0, 6.0 Hz, 1H, H-6b′), 3.29 (dt, J=9.6, 7.2 Hz, 1H, CH₂), 2.11-1.88 (m, 4H, CH₂), 1.55-1.49 (m, 4H, CH₂), 1.45 (s, 3H, CH₃), 1.35 (s, 3H, CH₃), 1.25 (bs, 82H, CH₂), 0.88 (t, J=10.8 Hz, 9H, CH₃×3); ¹³C NMR (150 MHz, CDCl₃) δ 172.4 (C), 138.6 (C), 138.4 (C), 138.3 (C), 135.0 (CH), 128.34 (CH×3), 128.32 (CH×3), 128.2 (CH×2), 127.9 (CH×2), 127.8 (CH), 127.8 (CH), 127.7 (CH), 127.4 (CH×2), 124.2 (CH), 108.3 (C), 99.3 (CH), 78.8 (CH), 75.9 (CH), 74.7 (CH₂), 74.6 (CH), 73.4 (CH₂), 73.0 (CH), 72.8 (CH₂), 71.7 (CH₂), 69.8 (CH₂), 69.6 (CH), 69.5 (CH), 49.0 (CH), 36.7 (CH₂), 31.9 (CH₂), 30.0 (CH₂), 29.7 (CH₂×28), 29.61 (CH₂×2), 29.60 (CH₂×2), 29.55 (CH₂×2), 29.49 (CH₂), 29.47 (CH₂), 29.40 (CH₂), 29.37 (CH₂×2), 28.0 (CH₃), 27.7 (CH₂), 26.1 (CH₂), 25.7 (CH₃), 25.4 (CH₂), 22.7 (CH₂), 14.1 (CH₃×3); HRMS (ESI, M+Na⁺) calculated for C₈₆H₁₄₃O₉NNa 1357.0655, found 1357.0661.

### Example 12

### preparation of Compound 11e ((2S,3S,4R)-1-O-(2,3,4-tri-O-benzyl-6-O-tridecyl-α-D-gala-ctopyranosyl)-2-hexacosanoylamino-3,4-O-isopropylidene-5-octadecen-1,3,4-triol)

To a solution of Compound 9 (149 mg, 0.13 mmol) in N,N-dimethylformamide (2 mL) were added 1-bromotridecane (65 μL, 0.25 mmol) and 60% sodium hydride (10 mg, 0.26 mmol) at 28° C. After complete addition, the reaction mixture was stirred for 8 h. Methanol was added to quench the reaction and concentrated in vacuo. The mixture was extracted with ethyl acetate (3×5 mL) and water (5 mL). The combined organic layers were dried over anhydrous MgSO₄, filtered, and concentrated in vacuo to give a residue. The residue was purified by column chromatography to afford Compound 11e (151 mg, 87%) as a yellow solid. R_{f} 0.52 (EtOAc/Hex=1/2.5); [α]²⁵_{D} +23.6 (c 0.1, CHCl₃); mp 49-50° C.; IR (CHCl₃) v 3591, 2919, 2851, 1660, 1511, 1467, 1043 cm⁻¹; ¹H NMR (600 MHz, CDCl₃) δ 7.41-7.27 (m, 15H, ArH), 6.20 (d, J=9.0 Hz, 1H, NH), 5.58 (td, J=10.8, 7.8 Hz, 1H, H-6), 5.42 (dd, J=10.8, 9.6 Hz, 1H, H-5), 4.94 (d, J=12.0 Hz, 1H, PhCH₂), 4.92 (d, J=3.6 Hz, 1H, H-1′), 4.85 (dd, J=9.0, 6.0 Hz, 1H, H-4), 4.81 (d, J=11.4 Hz, 1H, PhCH₂), 4.80 (d, J=11.4 Hz, 1H, PhCH₂), 4.75 (d, J=11.4 Hz, 1H, PhCH₂), 4.68 (d, J=12.0 Hz, 1H, PhCH₂), 4.62 (d, J=12.0 Hz, 1H, PhCH₂), 4.21 (dd, J=9.0, 5.4 Hz, 1H, H-3), 4.08-4.03 (m, 2H, H-2, H-2′), 4.00 (dd, J=10.8, 3.0 Hz, 1H, H-1a), 3.94 (bs, 1H, H-4′), 3.92-3.88 (m, 2H, H-3′, H-5′), 3.63 (dd, J=11.4, 2.4 Hz, 1H, H-1b), 3.45 (dd, J=9.6, 6.6 Hz, 1H, H-6a′), 3.39 (td, J=9.6, 7.2 Hz, 1H, CH₂), 3.34 (dd, J=9.0, 6.0 Hz, 1H, H-6b′), 3.29 (td, J=9.0, 7.2 Hz, 1H, CH₂), 2.10-1.87 (m, 4H, CH₂), 1.55-1.47 (m, 6H, CH₂), 1.45 (s, 3H, CH₃), 1.35 (s, 3H, CH₃), 1.25 (bs, 82H, CH₂), 0.88 (t, J=6.6 Hz, 9H, CH₃×3); ¹³C NMR (150 MHz, CDCl₃) δ 172.4 (C), 138.6 (C), 138.4 (C), 138.3 (C), 135.0 (CH), 129.5 (CH), 128.4 (CH×3), 128.3 (CH×3), 128.2 (CH×2), 127.9 (CH×2), 127.8 (CH), 127.7 (CH), 127.5 (CH), 127.4 (CH×2), 124.1 (CH), 108.3 (C), 99.3 (CH), 78.8 (CH), 76.8 (CH), 75.8 (CH), 74.7 (CH₂), 74.5 (CH), 73.4 (CH₂), 73.0 (CH), 72.8 (CH₂), 71.7 (CH₂), 69.8 (CH₂), 69.6 (CH), 69.5 (CH₂), 49.0 (CH), 36.7(CH₂), 31.9 (CH₂), 29.7 (CH₂×26), 29.6 (CH₂×2), 29.56 (CH₂×2), 29.50 (CH₂), 29.48 (CH₂), 29.42 (CH₂), 29.38 (CH₂×2), 28.0 (CH₃), 27.7 (CH₂), 26.1 (CH₂), 25.7 (CH₃), 25.4 (CH₂), 22.7 (CH₂), 14.1 (CH₃×3); HRMS (ESI, M+Na⁺) calculated for C₈₇H₁₄₅O₉NNa 1371.0812, found 1371.0806.

### Example 13

### Preparation of Compound 11f ((2S,3S,4R)-1-O-(2,3,4-tri-O-benzyl-6-eicosyl-α-D-galacto-pyranosyl)-2-hexacosanoylamino-3,4-O-isopropylidene-5-oc-tadecen-1,3,4-triol)

To a solution of Compound 9 (33 mg, 0.028 mmol) in N,N-dimethylformamide (1 mL) were added 1-bromoeicosane (20 mg, 0.06 mmol) and 60% sodium hydride (2 mg, 0.06 mmol) at 28° C. After complete addition, the reaction mixture was stirred for 12 h. Methanol was added to quench the reaction and concentrated in vacuo. The mixture was extracted with ethyl acetate (3×5 mL) and water (5 mL). The combined organic layers were dried over anhydrous MgSO₄, filtered, and concentrated in vacuo to give a residue. The residue was purified by column chromatography to afford Compound 11f (37 mg, 91%) as a yellow solid. R_{f} 0.68 (EtOAc/Hex=1/2.5); [α]²⁵_{D} +23.0 (c 0.4, CHCl₃); mp 56-58° C.; IR (CHCl₃) v 3342, 2919, 2851, 1649, 1538, 1468, 1056 cm⁻¹; ¹H NMR (600 MHz, CDCl₃) δ 7.41 (m, 15H, ArH), 6.22 (d, J=9.0 Hz, 1H, NH), 5.58 (td, J=10.8, 7.2 Hz, 1H, H-6), 5.42 (t, J=9.6 Hz, 1H, H-5), 4.94 (d, J=12.0 Hz, 1H, PhCH₂), 4.93 (d, J=4.2 Hz, 1H, H-1′), 4.85 (dd, J=9.0, 6.0 Hz, 1H, H-4), 4.81 (d, J=11.4 Hz, 1H, PhCH₂), 4.80 (d, J=11.4 Hz, 1H, PhCH₂), 4.75 (d, J=12.0 Hz, 1H, PhCH₂), 4.68 (d, J=12.0 Hz, 1H, PhCH₂), 4.62 (d, J=11.4 Hz, 1H, PhCH₂), 4.21 (dd, J=9.6, 6.0 Hz, 1H, H-3), 4.08-4.03 (m, 2H, H-2, H-2′), 3.99 (dd, J=12.0, 3.6 Hz, 1H, H-1a), 3.95 (bs, 1H, H-4′), 3.92-3.88 (m, 2H, H-3′, H-5′), 3.63 (dd, J=12.0, 2.4 Hz, 1H, H-1b), 3.45 (dd, J=6.6, 3.6 Hz, 1H, H-6a′), 3.39 (td, J=9.6, 7.2 Hz, 1H, CH₂), 3.34 (dd, J=9.0, 6.0 Hz, 1H, H-6b′), 3.30 (td, J=9.6, 7.2 Hz, 1H, CH₂), 2.11-1.87 (m, 4H, CH₂), 1.56-1.49 (m, 4H, CH₂), 1.45 (s, 3H, CH₃), 1.35 (s, 3H, CH₃), 1.25 (bs, 98H, CH₂), 0.88 (t, J=6.6 Hz, 9H, CH₃×3); ¹³C NMR (150 MHz, CDCl₃) δ 172.4 (C), 138.6 (C), 138.4 (C), 138.3 (C), 135.0 (CH), 128.37 (CH×2), 128.5 (CH×2), 128.3 (CH×2), 128.2 (CH×2), 127.9 (CH×2), 127.8 (CH), 127.7 (CH), 127.54 (CH), 127.45 (CH×2), 124.1 (CH), 108.3 (C), 99.3 (CH), 78.8 (CH), 76.8 (CH), 75.8 (CH), 74.7 (CH₂), 74.6 (CH), 73.4 (CH₂), 73.0 (CH), 72.8 (CH₂), 71.7 (CH₂), 69.8 (CH₂), 69.6 (CH), 69.5 (CH₂), 49.0 (CH), 36.7 (CH₂), 31.9 (CH₂), 29.7 (CH₂×38), 29.6 (CH₂×2), 29.58 (CH₂×2), 29.50 (CH₂), 29.48 (CH₂), 29.41 (CH₂), 29.36 (CH₂×2), 28.0 (CH₃), 27.7 (CH₂), 26.1 (CH₂), 25.7 (CH₃), 25.4 (CH₂), 22.7 (CH₂), 14.1 (CH₃×3); HRMS (ESI, M+Na⁺) calculated for C₉₄H₁₅₉O₉NNa 1469.1907, found 1469.1926.

### Example 14

### Preparation of compound 2c ((2S,3S,4R)-1-O-(6-O-hexyl-α-D-galactopyranosyl)-D-ribo-2-hexacosanoylamino-1,3,4-octa-decantriol)

Compound 11c (49 mg) was dissolved in a mixed solvent of MeOH/CHCl₃ (3/1 ratio, 4 mL) at 28° C. The Pd(OH)₂/C (49 mg, Degussa type) was added to the solution and followed by addition 2-3 drops of acetic acid, the reaction vessel was purged with hydrogen, and the mixture was stirred under 60 psi pressure at the same temperature for 5 h. The resulting solution was filtered through celite, the filtrate was concentrated in vacuo, and the residue was purified by column chromatography to afford Compound 2c (27 mg, 74%) as white solid. R_{f} 0.3 (MeOH/DCM=1/10); [α]²⁵_{D}+36.3 (c 0.1, CHCl₃); mp 70-72° C.; IR (CHCl₃) v 3279, 2920, 2851, 1642, 1036 cm⁻¹; ¹H NMR (600 MHz, C₅D₅N) δ 8.49 (d, J=8.4 Hz, 1H, NH), 5.53 (d, J=3.0 Hz, 1H, H-1′), 5.27-5.23 (m, 1H, H-2), 4.66 (dd, J=10.8, 5.4 Hz, 1H, H-1a), 4.63 (dd, J=9.6, 3.6 Hz, 1H, H-2′), 4.49 (t, J=6.6 Hz, 1H, H-5′), 4.42 (d, J=2.4 Hz, 1H, H-4′), 4.39 (dd, J=9.6, 3.6 Hz, 1H, H-1b), 4.38 (t, J=5.4 Hz, 1H, H-3′), 4.35-4.30 (m, 2H, H-3, H-4), 4.10 (dd, J=10.2, 6.6 Hz, 1H, H-6a′), 4.00 (dd, J=10.2, 6.6 Hz, 1H, H-6b′), 3.54-3.47 (m, 2H, CH₂), 2.46-2.43 (m, 2H, CH₂), 2.29-2.25 (m, 1H, CH₂), 1.94-1.86 (m, 2H, CH₂), 1.85-1.80 (m, 2H, CH₂), 1.71-1.67 (m, 2H, CH₂), 1.60-1.57(m, 2H, CH₂), 1.30 (bs, 48H, CH₂), 1.23 (bs, 23H, CH₂), 0.85 (t, J=6.6 Hz, 6H, CH₃×2), 0.82 (t, J=6.6 Hz, 3H, CH₃); ¹³C NMR (150 MHz, C₅D₅N) δ 173.1 (C), 100.5 (CH), 76.5 (CH), 72.4 (CH), 71.6 (CH₂), 71.4 (CH), 71.0 (CH₂), 70.8 (CH), 70.7 (CH), 70.1 (CH), 68.7 (CH₂), 51.3 (CH), 36.8 (CH₂), 34.2 (CH₂), 32.1 (CH₂), 31.9 (CH₂), 30.44 (CH₂), 30.36 (CH₂), 30.2 (CH₂ X 2), 30.00 (CH₂×19), 29.92 (CH₂×4), 29.83 (CH₂), 29.77 (CH₂), 29.6 (CH₂), 26.5 (CH₂), 26.4 (CH₂), 26.1 (CH₂×2), 22.93 (CH₂×2), 22.87 (CH₂), 14.3 (CH₃×2), 14.2 (CH₃); HRMS (ESI, M+Na⁺) calculated for C₅₆H₁₁₁O₉NNa 964.8151, found 964.8160.

### Example 15

### Preparation of Compound 2d ((2S,3S,4R)-1-O-(6-O-dodecyl-α-D-galactopyranosyl)-D-ribo-2-hexacosanoylamino-1,3,4-oct-adecantriol)

Compound 11d (17 mg) was dissolved in a mixed solvent of MeOH/CHCl₃ (3/1 ratio, 2 mL) at 28° C. The Pd(OH)₂/C (17 mg, Degussa type) was added to the solution and followed by addition 2-3 drops of acetic acid, the reaction vessel was purged with hydrogen, and the mixture was stirred under 60 psi pressure at the same temperature for 5 h. The resulting solution was filtered through celite, the filtrate was concentrated in vacuo, and the residue was purified by column chromatography to afford Compound 2d (11 mg, 94%) as white solid. R_{f} 0.21 (MeOH/DCM=1/10); [α]²⁵_{D} +46.7 (c 0.05, CHCl₃); mp 92-93° C.; IR (CHCl₃) v 3308, 2920, 2851, 1647, 1036 cm⁻¹; ¹H NMR (600 MHz, C₅D₅N) δ 8.51 (d, J=9.0 Hz, 1H, NH), 6.49 (bs, 1H, OH), 6.44 (bs, 1H, OH), 6.12 (bs, 1H, OH), 5.53 (d, J=3.0 Hz, 1H, H-1′), 5.26-5.23 (m, 1H, H-2), 4.67-4.62 (m, 2H, H-1a, H-2′), 4.50 (t, J=6.0 Hz, 1H, H-5′), 4.42-4.38 (m, 3H, H-1b, H-3′, H-4′), 4.34-4.31 (m, 2H, H-3, H-4), 4.11 (dd, J=10.2, 6.0 Hz, 1H, H-6a′), 4.02 (dd, J=9.6, 6.0 Hz, 1H, H-6b′), 3.57-3.50 (m, 2H, CH₂), 2.46-2.43 (m, 2H, CH₂), 2.28-2.27 (m, 1H, CH₂), 1.92-1.81 (m, 6H, CH₂), 1.71-1.61 (m, 8H, CH₂), 1.30-1.24 (bs, 77H, CH₂), 0.86 (t, J=6.6 Hz, 9H, CH₃ X 2); ¹³C NMR (150 MHz, C₅D₅N) δ 173.1 (C), 101.5 (CH), 76.5 (CH), 72.4 (CH), 71.7 (CH₂), 71.4 (CH), 71.0 (CH₂), 70.8 (CH), 70.7 (CH), 70.1 (CH), 68.8 (CH₂), 37.6 (CH₂), 37.3 (CH₂), 36.8 (CH₂), 34.2 (CH₂), 33.9 (CH₂), 33.0 (CH), 32.1 (CH₂×3), 30.5 (CH₂), 30.4 (CH₂), 30.3 (CH₂×2), 30.2 (CH₂), 30.0 (CH₂×16), 29.92 (CH₂×4), 29.85 (CH₂×2), 29.8 (CH₂×2), 29.6 (CH₂×3), 29.4 (CH₂), 27.0 (CH₂), 26.6 (CH₂), 26.4 (CH₂), 22.9 (CH₂×3), 14.3 (CH₃×3); HRMS (ESI, M+H⁺) calculated for C₆₂H₁₂₄O₉N 1026.9271, found 1026.9285.

### Example 16

### Preparation of Compound 2e ((2S,3S,4R)-1-O-(6-O-tridecyl-α-D-galactopyranosyl)-D-ribo-2-hexacosanoylamino-1,3,4-oct-adecantriol)

Compound 11e (22 mg) was dissolved in a mixed solvent of MeOH/CHCl₃ (3/1 ratio, 2 mL) at 28° C. The Pd(OH)₂/C (22 mg, Degussa type) was added to the solution and followed by addition 2-3 drops of acetic acid, the reaction vessel was purged with hydrogen, and the mixture was stirred under 60 psi pressure at the same temperature for 5 h. The resulting solution was filtered through celite, the filtrate was concentrated in vacuo, and the residue was purified by column chromatography to afford Compound 2e (15.7 mg, 91%) as white solid. R_{f} 0.24 (MeOH/DCM=1/10); [α]²⁵_{D} +20.6 (c 0.4, CHCl₃); mp 88-90° C.; IR (CHCl₃) v 3331, 2920, 2851, 1648, 1032 cm⁻¹; ¹H NMR (600 MHz, C₅H₅N) δ 8.53 (d, J=8.4 Hz, 1H, NH), 6.50 (bs, 1H, OH), 6.12 (bs, 1H, OH), 5.52 (d, J=3.6 Hz, 1H, H-1′), 5.25-5.21 (m, 1H, H-2), 4.65 (dd, J=10.8, 4.8 Hz, 1H, H-1a), 4.62 (dd, J=9.6, 3.6 Hz, 1H, H-2′), 4.49 (t, J=6.6 Hz, 1H, H-5′), 4.41-4.38 (m, 3H, H-1b, H-3′, H-4′), 4.36-4.30 (m, 2H, H-3, H-4), 4.10 (dd, J=10.2, 6.6 Hz, 1H, H-6a′), 4.01 (dd, J=10.2, 6.6 Hz, 1H, H-6b′), 3.57-3.50 (m, 2H, CH₂), 2.46-2.43 (m, 2H, CH₂), 2.30-2.24 (m, 1H, CH₂), 2.07-1.80 (m, 8H, CH₂), 1.71-1.60 (m, 7H, CH₂), 1.30 (bs, 23H, CH₂), 1.25 (bs, 23H, CH₂), 1.24 (bs, 32H, CH₂), 0.85 (t, J=6.6 Hz, 9H, CH₃×3); ¹³C NMR (150 MHz, C₅H₅N) δ 173.1 (C), 101.4 (CH), 76.4 (CH), 72.4 (CH), 71.7 (CH₂), 71.4 (CH), 71.0 (CH₂), 70.8 (CH), 70.7 (CH), 70.1 (CH), 68.7 (CH₂), 51.3 (CH), 37.3 (CH₂), 36.8 (CH₂), 34.1 (CH₂), 32.1 (CH₂×2), 30.4 (CH₂), 30.3 (CH₂), 30.2 (CH₂), 30.0 (CH₂×29), 29.85 (CH₂×2), 29.77 (CH₂), 29.6 (CH₂×2), 27.4 (CH₂), 27.0 (CH₂), 26.5 (CH₂), 26.4 (CH₂), 22.9 (CH₂×2), 14.3 (CH₃×3); HRMS (ESI, M+Na⁺) calculated for C₆₃H₁₂₅O₉NNa 1062.92466, found 1062.92475.

### Example 17

### Preparation of Compound 2f ((2S,3S,4R)-1-O-(6-O-eicosanyl-α-D-galactopyranosyl)-D-ribo-2-hexacosanoylamino-1,3,4-oc-tadecantriol)

Compound 11f (81 mg) was dissolved in a mixed solvent of MeOH/CHCl₃ (3/1 ratio, 4 mL) at 28° C. The Pd(OH)₂/C (81 mg, Degussa type) was added to the solution and followed by addition 2-3 drops of acetic acid, the reaction vessel was purged with hydrogen, and the mixture was stirred under 60 psi pressure at the same temperature for 5 h. The resulting solution was filtered through celite, the filtrate was concentrated in vacuo, and the residue was purified by column chromatography to afford Compound 2f (23 mg, 35%) as white solid. R_{f} 0.38 (MeOH/DCM=1/10); [α]²⁵_{D} +50.0(c 0.12, CHCl₃); mp 98-100° C.; IR (CHCl₃) 3272, 2918, 2850, 1649, 1033 cm⁻¹; ¹H NMR (600 MHz, C₅H₅N) δ 8.46 (d, J=8.4 Hz, 1H, NH), 5.53 (d, J=4.2 Hz, 1H, H-1′), 5.23-5.21 (m Hz, 1H, H-2), 4.66 (dd, J=10.8, 5.4 Hz, 1H, H-1a), 4.63 (dd, J=9.6, 4.2 Hz, 1H, H-2′), 4.51 (t, J=6.6 Hz, 1H, H-5′), 4.43-4.39 (m, 3H, H-1b, H-3′, H-4′), 4.35-4.31 (m, 2H, H-3, H-4), 4.11 (dd, J=9.6, 6.0 Hz, 1H, H-6a′), 4.03 (dd, J=9.6, 6.0 Hz, 1H, H-6b′), 3.59-3.51 (m, 2H, CH₂), 2.48-2.43 (m, 2H, CH₂), 2.31-2.26 (m, 1H, CH₂), 1.95-1.81 (m, 5H, CH₂), 1.72-1.63 (m, 5H, CH₂), 1.31 (bs, 36H, CH₂), 1.28 (bs, 21H, CH₂), 1.25 (bs, 40H, CH₂), 0.87-0.84 (m, 9H, CH₃×3); ¹³C NMR (150 MHz, C₅H₅N) δ 173.1 (C), 101.5 (CH), 76.5 (CH), 72.5 (CH), 71.7 (CH₂), 71.4 (CH), 71.0 (CH₂), 70.8 (CH), 70.7 (CH), 70.2 (CH), 68.8 (CH₂), 51.3 (CH), 36.7 (CH₂), 34.2 (CH₂), 32.1 (CH₂×4), 30.4 (CH₂), 30.3 (CH₂), 30.2 (CH₂), 30.0 (CH₂×27), 29.94 (CH₂×6), 29.86 (CH₂), 29.8 (CH₂), 29.6 (CH₂×4), 26.6 (CH₂), 26.5 (CH₂), 26.4 (CH₂), 22.9 (CH₂×4), 14.3 (CH₃×3); HRMS (CI, M+H⁺) calculated for C₇₀H₁₄₀O₉N 1139.0523, found 1139.0511.

### Example 18

### Preparation of Compound 11g ((2S,3S,4R)-1-O-(2,3,4-tri-O-benzyl-6-O-diphenylphospho-ryl-α-D-galactopyranosyl)-2-hexa-cosanoylamino-3,4-O-iso-propylidene-5-octadecen-1,3,4-triol)

To a solution of Compound 9 (200 mg, 0.17 mmol) and diphenylphosphoryl azide (222 μL, 1.03 mmol) in dichloromathane (2.0 mL) at 0° C. was added 1,8-diazabicyclo[5.4.0]undec-7-ene (DBU, 146 μL, 0.98 mmol), the reaction mixture was stirred at the same temperature for 2 h. Water (3.0 mL) was added to quench the reaction and the mixture was extracted with dichloromathane (2×3 mL). The combined organic layers were washed with brine, dried over anhydrous MgSO₄, filtered, and concentrated in vacuo to give a residue. The residue was purified by column chromatography to afford Compound 11g (224 mg, 93%) as white solid. R_{f} 0.53 (EtOAc/Hex=1/3); [α]²⁵_{D} +27.3 (c 1.0, CHCl₃); mp 58-60° C.; IR (CHCl₃) v 3318, 2919, 2850, 1645 cm⁻¹; ¹H NMR (600 MHz, CDCl₃) δ 7.40-7.17 (m, 25H, ArH), 6.01-5.99 (m, 1H, NH), 5.59-5.55 (m, 1H, H-6), 5.43-5.39 (m, 1H, H-5), 5.03 (d, J=3.0 Hz, 1H, H-1′), 4.93 (d, J=11.4 Hz, 1H, CH₂Ph), 4.86-4.83 (m, 1H, H-4), 4.80 (d, J=11.4 Hz, 1H, CH₂Ph), 4.78 (d, J=11.4 Hz, 1H, CH₂Ph), 4.74 (d, J=11.4 Hz, 1H, CH₂Ph), 4.68 (d, J=11.4 Hz, 1H, CH₂Ph), 4.52 (d, J=11.4 Hz, 1H, CH₂Ph), 4.33-4.29 (m, 1H, H-6a′), 4.22-4.16 (m, 2H, H-3, H-6b′), 4.08-4.04 (m, 2H, H-2, H-2′), 4.00 (t, J=6.6 Hz, 1H, H-5′), 3.90-3.89 (m, 2H, H-3′, H-4′), 3.81 (dd, J=11.4, 2.4 Hz, 1H, H-1a), 3.62 (dd, J=10.8, 1.8 Hz, 1H, H-1b), 2.08-1.86 (m, 6H, CH₂), 1.53-1.49 (m, 2H, CH₂), 1.43 (s, 3H, CH₃), 1.35 (s, 3H, CH₃), 1.31-1.23 (m, 62H, CH₂), 0.88 (t, J=7.2 Hz, 6H, CH₃); ¹³C NMR (150 MHz, CDCl₃) δ 172.2 (C), 150.4 (t, C×2), 138.4 (C), 138.2 (C), 138.1 (C), 135.1 (CH), 129.80 (d, CH×4), 128.40 (CH×2), 128.39 (CH×2), 128.3 (CH×2), 128.2 (CH×2), 127.92 (CH×2), 127.87 (CH), 127.74 (CH), 127.65 (CH), 127.4 (CH×2), 125.5 (d, CH×2), 124.0 (CH), 120.0 (d, CH×4), 108.3 (C), 98.6 (CH), 78.6 (CH), 76.6 (CH), 76.0 (CH), 74.7 (CH₂), 74.0 (CH), 73.6 (CH₂), 73.1 (CH), 72.8 (CH₂), 69.3 (d, CH), 68.4 (CH₂), 67.3 (t, CH₂), 48.9 (CH), 36.7 (CH₂), 31.9 (CH₂×2), 29.7 (CH₂×22), 29.6 (CH₂×2), 29.5 (CH₂×2), 29.4 (CH₂×2), 27.9 (CH₃), 27.7 (CH₂), 25.6 (CH₃), 25.4 (CH₂), 22.7 (CH₂×2), 14.1 (CH₃×2); HRMS (ESI, M+H⁺) calculated for C₈₆H₁₂₉O₁₂NP 1398.9247, found 1398.9257.

### Example 19

### Preparation of Compound 12 ((2S,3S,4R)-1-O-(2,3,4-tri-O-benzyl-6-O-diphenylphospho-ryl-α-D-galactopyranosyl)-2-hexa-cosanoylamino-5-octade-cen-1,3,4-triol)

To a solution of Compound 11 g (41 mg, 0.03 mmol) in 1,4-dioxane (800 μL) was added 75% H₂SO₄ (20 μL) and stirred for 30 min. Saturated sodium bicarbonate was added to quench the reaction, and the reaction was extracted with ethyl acetate (2×2 mL). The organic layer was dried over anhydrous MgSO₄, filtered, and concentrated in vacuo. The residue was purified by column chromatography to obtain Compound 12 (30 mg, 74%) as white solid. R_{f} 0.24 (EtOAc/Hex=1/2); [α]²⁵_{D} +20.3 (c 0.9, CHCl₃); mp 52° C.; IR (CHCl₃) v 3337, 2919, 2850, 1614, 1543, 1191, 1026 cm⁻¹; ¹H NMR (600 MHz, CDCl₃) δ 7.38-7.15 (m, 25H, ArH), 6.23 (d, J=8.4 Hz, 1H, NH), 5.62-5.58 (m, 1H, H-6), 5.43-5.39 (m, 1H, H-5), 4.91 (d, J=11.4 Hz, 1H, CH₂Ph), 4.89 (d, J=3.6 Hz, 1H, H-1′), 4.85 (d, J=12.0 Hz, 1H, CH₂Ph), 4.78 (d, J=11.4 Hz, 1H, CH₂Ph), 4.72 (d, J=11.4 Hz, 1H, CH₂Ph), 4.71 (d, J=11.4 Hz, 1H, CH₂Ph), 4.50 (d, J=11.4 Hz, 1H, CH₂Ph), 4.46-4.45 (m, 1H, H-4), 4.31-4.26 (m, 1H, H-6a′), 4.22-4.18 (m, 1H, H-2), 4.16-4.11 (m, 1H, H-6b′), 4.06-4.03 (m, 2H, H-2′, H-5′), 3.86-3.84 (m, 2H, H-3′, H-4′), 3.79 (dd, J=10.8, 4.2 Hz, 1H, H-1a), 3.70 (dd, J=10.8, 3.6 Hz, 1H, H-1b), 3.57-3.54 (m, 1H, H-3), 3.44 (bs, 1H, OH), 3.04 (bs, 1H, OH), 2.12-1.98 (m, 4H, CH₂), 1.58-1.56 (m, 2H, CH₂), 1.34-1.24 (m, 64H, CH₂), 0.88 (t, 6H, CH₃); ¹³C NMR (150 MHz, CDCl₃) δ 173.0 (C), 150.3 (t, C×2), 138.1 (C), 138.0 (C), 137.6 (C), 134.9 (CH), 129.8 (CH×4), 128.5 (CH×2), 128.4 (CH×2), 128.3 (CH×2), 128.22 (CH×2), 128.18 (CH×2), 128.1 (CH), 128.0 (CH), 127.8 (CH), 127.7 (CH), 127.4 (CH×2), 125.5 (d, CH×2), 120.0 (d, CH×4), 98.8 (CH), 78.8 (CH), 75.7 (CH), 75.3 (CH), 74.6 (CH₂), 74.0 (CH₂), 73.8 (CH), 73.0 (CH₂), 69.4 (d, CH), 68.9 (CH₂), 68.8 (CH), 67.3 (CH₂), 49.8 (CH), 36.6 (CH₂), 31.9 (CH₂×2), 29.7 (CH₂×16), 29.62 (CH₂×4), 29.57 (CH₂), 29.5 (CH₂), 29.4 (CH₂×2), 29.3 (CH₂×4), 28.0 (CH₂), 25.6 (CH₂), 22.7 (CH₂×2), 14.1 (CH₃×2); HRMS (ESI, M+H⁺) calculated for C₈₃H₁₂₅O₁₂NP 1358.8934, found 1358.8967.

### Example 20

### Preparation of Compound 2g (2S,3S,4R)-1-O-(6-O-phospho-α-D-galactopyranosyl)-D-ribo-2-hexacosanoylamino-1,3,4-oc-tadecantriol, phosphoric acid)

Compound 12 (140 mg) was dissolved in a mixed solvent of MeOH/CHCl₃ (3/1 ratio, 2.0 mL) at room temperature. Pd(OH)₂/C (100 mg, Degussa type) was added to the solution, the reaction vessel was purged with hydrogen, and the mixture was stirred under 60 psi pressure at the same temperature for 1 d. The resulting solution was filtered through celite, the filtrate was concentrated in vacuo. The residue was dissolved in MeOH/CHCl₃ (3/1 ratio, 2.0 mL), Adam's catalyst (PtO₂, 70 mg) was added, and the reaction vessel was purged with hydrogen, and the mixture was stirred under 60 psi pressure at the same temperature for 1 d. The catalyst was removed by filtration, and the filtrate was concentrated in vacuo, filtered, and washed the solid to afford the crude Compound 2g as white solid. [α]²²_{D} +39.9 (c 0.4, CHCl₃/MeOH); mp 182° C.; IR (KBr) v 2918, 2849, 1742, 1466, 1173 cm⁻¹; ¹H NMR (600 MHz, d-pyridine) δ 8.61 (d, J=8.4 Hz, 1H, NH), 5.46 (d, J=3.6 Hz, 1H, H-1′), 5.22-5.20 (m, 1H, H-2), 4.94 (dd, J=16.2, 9,6 Hz, 1H, H-6a′), 4.76 (dd, J=15.6, 9,0 Hz, 1H, H-6b′), 4.70 (t, J=6.0 Hz, 1H, H-5′), 4.63 (dd, J=10.8, 4.8 Hz, 1H, H-1a), 4.58 (dd, J=10.2, 3.6 Hz, 1H, H-2′), 4.52 (bs, 1H, H-3′), 4.38-4.24 (m, 4H, H-1b, H-3, H-4, H-4′), 2.46 (t, J=7.2 Hz, 2H, CH₂), 2.28-2.23 (m, 1H, H-5a), 1.94-1.87 (m, 1H, H-5b), 1.83-1.76 (m, 2H, CH₂), 1.71-1.67 (m, 2H, CH₂), 1.39-1.12 (m, 66H, CH₂), 0.84 (m, 6H, CH₃); ¹³C NMR (150 MHz, C₅D₅N) δ 173.4 (C), 101.4 (CH), 76.5 (CH), 72.3 (CH), 71.1 (CH), 71.0 (CH), 70.2 (CH), 69.9 (CH), 68.5 (CH₂), 65.3 (CH₂), 51.6 (CH), 36.8 (CH₂), 34.2 (CH₂), 32.09 (CH₂×2), 32.08 (CH₂×2), 30.4 (CH₂), 30.2 (CH₂), 30.0 (CH₂×19), 29.83 (CH₂), 29.75 (CH₂), 29.60 (CH₂×2), 29.58 (CH₂×2), 26.5 (CH₂), 26.4 (CH₂), 22.9 (CH₂×2), 14.3 (CH₃×2); HRMS (ESI, M+H⁺) calculated for C₅₀H₉₉O₁₂NP 936.6899, found 936.6869.

### Example 21

### Preparation of Compound 13 ((2S,3S,4R)-1-O-(2,3,4-tri-O-benzyl-6-O-sulfo-α-D-galacto-pyranosyl)-2-hexacosanoylamino-5-octadecen-1,3,4-triol, sodium salt)

To a solution of Compound 9 (92 mg, 0.08 mmol) and SO₃/TMA (55 mg, 0.40 mmol) in DMF (1.5 mL), and the mixture was kept stirring for 12 h. Sodium bicarbonate (100 mg, 1.19 mmol) in water (3.0 mL) was added to the solution and stirred for 30 min., filtered to afford the Compound 13 (100 mg, quant.) as white solid. R_{f} 0.36 (EtOAc); [α]²⁴_{D} +32.1 (c 0.5, CHCl₃); IR (CHCl₃) v 3312, 2919, 2851, 1644, 1543, 1219 cm⁻¹; ¹H NMR (600 MHz, CDCl₃) δ 7.18 (m, 15H, ArH), 6.06 (d, J=9.0 Hz, 1H, NH), 5.54 (td, J=10.8, 7.2 Hz, 1H, H-6), 5.37 (t, J=10.2 Hz, 1H, H-5), 5.04 (d, J=3.6 Hz, 1H, H-1′), 4.87 (d, J=10.8 Hz, 1H, PhCH₂), 4.86-4.85 (m, J=5.4 Hz, 1H, H-4), 4.73-4.71 (m, 3H, PhCH₂), 4.66 (d, J=11.4 Hz, 1H, PhCH₂), 4.60 (d, J=10.8 Hz, 1H, PhCH₂), 4.19-4.13 (m, 3H, H-3, H-6a′, H-6b′), 4.10-4.06 (m, 2H, H-2, H-5′), 4.03-4.01 (m, 2H, H-2′, H-4′), 3.86 (dd, J=10.2, 2.4 Hz, 1H, H-3′), 3.82-3.80 (m, 1H, H-1a), 3.70-3.68 (m, 1H, H-1b), 2.11-2.04 (m, 1H, H-7a), 1.98-1.88 (m, 3H, H-7b, CH₂), 1.46-1.45 (m, 2H, CH₂), 1.44 (s, 3H, CH₃), 1.34 (s, 3H, CH₃), 1.29-1.20 (m, 64H, CH₂), 0.88 (t, J=6.6 Hz, 6 H, CH₃); ¹³C NMR (150 MHz, CDCl₃) δ 173.7 (C), 138.6 (C), 138.32 (C), 138.25 (C), 135.4 (CH), 128.3 (CH×8), 127.9 (CH×2), 127.7 (CH), 127.6 (CH), 127.50 (CH×2), 127.45 (CH), 123.8 (CH), 108.5 (C), 97.4 (CH), 78.6 (CH), 76.5 (CH), 75.6 (CH), 74.7 (CH₂), 74.6 (CH), 73.0 (CH₂), 72.9 (CH), 72.4 (CH₂), 69.0 (CH), 67.6 (CH₂), 66.7 (CH₂), 48.8 (CH), 36.8 (CH₂), 31.9 (CH₂×2), 29.8 (CH₂×8), 29.7 (CH₂×12), 29.66 (CH₂), 29.63 (CH₂), 29.59 (CH₂), 29.56 (CH₂), 29.5 (CH₂×2), 29.38 (CH₂), 29.36 (CH₂), 28.0 (CH₃), 27.7 (CH₂), 25.7 (CH₃), 25.5 (CH₂), 22.7 (CH₂×2), 14.1 (CH₃×2); HRMS (ESI, M+H⁺) calculated for C₇₄H₁₁₉O₁₂NNaS 1268.8345 found 1268.8296.

### Example 22

### Preparation of Compound 14 ((2S,3S,4R)-1-O-(2,3,4-tri-O-benzyl-6-O-tert-butyldipheny-lsilyl-α-D-galactopyranosyl)-2-he-xacosanoylamino-5-octade-cen-1,3,4-triol)

To a solution of Compound 8 (690 mg, 0.49 mmol) in 1,4-dioxane (1.3 mL) was added 75% H₂SO₄ (345 μL) and was kept stirring for 30 min. Saturated sodium bicarbonate was added to quench the reaction, and the reaction was extracted with ethyl acetate (3×3 mL). The organic layer was dried over MgSO₄, filtered, and concentrated in vacuo. The residue was purified by column chromatography to obtain Compound 14 (432 mg, 64%) as colorless oil. R_{f} 0.21 (EtOAc/Hex=1/3); [α]²⁵_{D} +21.2 (c 1.6, CHCl₃); IR (CHCl₃) v 3411, 2924, 2853, 1650, 1464, 1091 cm⁻¹; ¹H NMR (600 MHz, CDCl₃) δ 7.60-7.20 (m, 25H, ArH), 6.24 (d, J=8.4 Hz, 1H, NH), 5.62-5.58 (m, 1H, H-6), 5.43-5.40 (m, 1H, H-5), 4.93 (d, J=10.8 Hz, 1H, PhCH₂), 4.89 (d, J=3.6 Hz, 1H, H-1′), 4.87 (d, J=11.4 Hz, 1H, PhCH₂), 4.82 (d, J=12.0 Hz, 1H, PhCH₂), 4.77 (d, J=12.0 Hz, 1H, PhCH₂), 4.71 (d, J=11.4 Hz, 1H, PhCH₂), 4.57 (d, J=10.8 Hz, 1H, PhCH₂), 4.46 (t, J=6.0 Hz, 1H, H-4), 4.25-4.22 (m, 1H, H-2), 4.02 (dd, J=10.2, 3.6 Hz, 1H, H-2′), 4.02 (d, 1H, J=2.4 Hz, H-4′), 3.88 (dd, J=10.2, 2.4 Hz, 1H, H-3′), 3.82 (dd, J=10.2, 4.2 Hz, 1H, H-1a), 3.76-3.71 (m, 3H, H-1b, H-5′, H-6a′), 3.68 (dd, J=9.6, 5.4 Hz, 1H, H-6b′), 3.55 (dd, J=10.8, 6.6 Hz, 1H, H-3), 3.50 (d, J=7.6 Hz, 1H, 3-OH), 2.80 (s, 1H, 4-OH), 2.14-1.98 (m, 4H, CH₂), 1.60-1.55 (m, 2H, CH₂), 1.34-1.25 (m, 64H, CH₂), 1.04 (s, 9H, CH₃), 0.88 (t, J=7.2 Hz, 6H, CH₃×2); ¹³C NMR (150 MHz, CDCl₃) δ 172.7 (C), 138.41 (C), 138.35 (C), 137.6 (C), 135.4 (CH×4), 135.0 (CH), 133.2 (C), 130.0 (C), 129.8 (CH), 129.7 (CH), 128.5 (CH×2), 128.4 (CH×2), 128.3 (CH×2), 128.2 (CH×2), 128.0 (CH), 127.9 (CH×3), 127.73 (CH×2), 127.71 (CH×2), 127.6 (CH), 127.5 (CH), 127.4 (CH×2), 98.7 (CH), 79.3 (CH), 75.9 (CH), 75.5 (CH), 74.8 (CH₂), 74.4 (CH), 74.2 (CH₂), 72.7 (CH₂), 71.5 (CH), 69.1 (CH), 68.7 (CH₂), 62.3 (CH₂), 49.3 (CH), 36.7 (CH₂), 31.9 (CH₂×2), 29.7 (CH₂×17), 29.64 (CH₂×2), 29.61 (CH₂), 29.58 (CH₂), 29.57 (CH₂), 29.5 (CH₂), 29.38 (CH₂×2), 29.35 (CH₂×3), 28.0 (CH₂), 26.8 (CH₃×3), 25.7 (CH₂), 22.7 (CH₂×2), 19.1 (C), 14.1 (CH₃×2); HRMS (ESI, M+H⁺) calculated for C₈₇H₁₃₄O₉NSi 1364.9822, found 1364.9845.

### Example 23

### Preparation of Compound 15 ((2S,3S,4R)-1-O-(2,3,4-tri-O-benzyl-6-O-tert-butyldiphenyl-silyl-α-D-galactopyranosyl)-3,4-di-O-benzyl-2-hexacosanoyl-amino-5-octadecen-1,3,4-triol)

To a solution of Compound 14 (80.5 mg, 0.06 mmol) and benzyl bromide (18 μL, 0.15 mmol) in tetrahydrofuran (1.0 mL) at 0° C. was added 60% sodium hydride (6.0 mg, 0.15 mmol). After completion of addition, the reaction mixture was brought to room temperature and stirred for 4 h. Water (3 mL) was added to quench the reaction and the mixture was extracted with ethyl acetate (2×3 mL). The combined organic layers were washed with brine, dried over anhydrous MgSO₄, filtered, and concentrated in vacuo to give a residue. The residue was purified by column chromatography to obtain Compound 15 (62 mg, 68%) as colorless oil. R_{f} 0.43 (EtOAc/Hex=1/7); [α]²⁵_{D} +15.4 (c 0.9, CHCl₃); IR (CHCl₃) v 2924 2853, 1680, 1498, 1456, 1095 cm⁻¹; ¹H NMR (600 MHz, CDCl₃) δ 7.61-7.20 (m, 35H, ArH), 5.96 (d, J=8.4 Hz, 1H, NH), 5.75-5.70 (m, 1H, H-6), 5.47 (t, J=10.2 Hz, 1H, H-5), 4.95 (d, J=10.8 Hz, 1H, PhCH₂), 4.84 (d, J=3.6 Hz, 1H, H-1′), 4.82 (d, J=12.0 Hz, 1H, PhCH₂), 4.75 (d, J=12.0 Hz, 1H, PhCH₂), 4.74 (d, J=12.0 Hz, 1H, PhCH₂), 4.72 (d, J=12.0 Hz, 1H, PhCH₂), 4.63 (d, J=11.4 Hz, 1H, PhCH₂), 4.564 (d, J=11.4 Hz, 1H, PhCH₂), 4.558 (d, J=12.0 Hz, 1H, PhCH₂), 4.51 (d, J=11.4 Hz, 1H, PhCH₂), 4.31-4.26 (m, 2H, H-2, H-4), 4.27 (d, J=12.0 Hz, 1H, PhCH₂), 4.05-4.01 (m, 2H, H-2′, H-4′), 3.92 (dd, J=10.2, 3.0 Hz, 1H, H-3′), 3.84-3.81 (m, 1H, H-3), 3.78-3.65 (m, 5H, H-1a, H-1b, H-5′, H-6a′, H-6b′), 2.00-1.81 (m, 6H, CH₂), 1.49-1.45 (m, 2H, CH₂), 1.30-1.20 (m, 62H, CH₂), 1.04 (s, 9H, CH₃), 0.88 (t, J=7.2 Hz, 6H, CH₃×2); ¹³C NMR (150 MHz, CDCl₃) δ 172.6 (C), 138.70 (C), 138.66 (C), 138.60 (C), 138.57 (C), 138.3 (C), 136.7 (CH), 135.5 (CH×4), 133.2 (C), 133.0 (C), 129.73 (CH), 129.67 (CH), 128.33 (CH×2), 128.29 (CH×2), 128.2 (CH×4), 128.1 (CH×2), 127.9 (CH×4), 127.73 (CH×2), 127.70 (CH×4), 127.6 (CH×3), 127.5 (CH), 127.44 (CH), 127.39 (CH), 127.37 (CH×3), 126.0 (CH), 98.6 (CH), 80.1 (CH), 79.1 (CH), 76.7 (CH), 74.9 (CH), 74.85 (CH), 74.83 (CH₂), 73.6 (CH₂), 73.4 (CH₂), 72.8 (CH₂), 71.1 (CH), 69.7 (CH₂), 67.1 (CH₂), 62.2 (CH₂), 50.2 (CH), 36.8 (CH₂), 31.9 (CH₂×2), 29.7 (CH₂×19), 29.64 (CH₂×2), 29.61 (CH₂×2), 29.5 (CH₂×2), 29.41 (CH₂), 29.36 (CH₂), 29.35 (CH₂), 28.0 (CH₂), 26.9 (CH₃×3), 25.7 (CH₂), 22.7 (CH₂×2), 19.1 (C), 14.1 (CH₃×2); HRMS (ESI, M+H⁻) calculated for C₁₀₁H₁₄₆O₉NSi 1545.0761, found 1545.0786.

### Example 24

### Preparation of Compound 16 (2S,3S,4R)-1-O-(2,3,4-tri-O-benzyl-α-D-galactopyranosyl)-3,4-di-O-benzyl-2-hexacosanoylamino-5-octadecen-1,3,4-triol)

To a solution of Compound 15 (111 mg, 0.07 mmol) in tetrahydrofuran (1.1 mL) was added 1.0 M solution of tetrabutylammonium fluoride in tetrahydrofuran (140 μL, 0.14 mmol) and stirred for 12 h. Water (2 mL) was added to quench the reaction and the mixture was extracted with ethyl acetate (2×2 mL). The combined organic layers were washed with brine, dried over anhydrous MgSO₄, filtered, and concentrated in vacuo to give a residue. The residue was purified by column chromatography to afford Compound 16 (84 mg, 90%) as white solid. R_{f} 0.31 (EtOAc/Hex=1/3); [α]²⁵_{D} −18.1 (c 1.0, CHCl₃); mp 64° C.; IR (CHCl₃) v 3334, 2921, 2851, 1639, 1538, 1455, 1056 cm⁻¹; ¹H NMR (400 MHz, CDCl₃) δ 7.38-7.25 (m, 25H, ArH), 5.82 (d, J=9.2 Hz, 1H, NH), 5.78-5.72 (m, 1H, H-6), 5.46 (t, J=10.0 Hz, 1H, H-5), 4.94 (d, J=11.2 Hz, 1H, PhCH₂), 4.84 (d, J=3.8 Hz, 1H, H-1′), 4.81 (d, J=11.6 Hz, 1H, PhCH₂), 4.79 (d, J=11.6 Hz, 1H, PhCH₂), 4.71 (d, J=12.0 Hz, 1H, PhCH₂), 4.67 (d, J=11.2 Hz, 1H, PhCH₂), 4.64 (d, J=11.2 Hz, 1H, PhCH₂), 4.63 (d, J=11.6 Hz, 1H, PhCH₂), 4.59 (d, J=12.0 Hz, 1H, PhCH₂), 4.50-4.45 (m, 1H, H-2), 4.45 (d, J=11.8 Hz, 1H, PhCH₂), 4.29 (d, J=11.8 Hz, 1H, PhCH₂), 4.28-4.25 (m, 1H, H-4), 4.02 (dd, J=9.6, 3.6 Hz, 1H, H-2′), 3.93 (dd, J=11.6, 8.0 Hz, 1H, H-1a), 3.85-3.82 (m, 2H, H-3′, H-4′), 3.78 (dd, J=11.6, 3.8 Hz, 1H, H-1b), 3.73-3.65 (m, 2H, H-5′, H-6a′), 3.58 (t, J=4.4 Hz, 1H, H-3), 3.50-3.45 (m, 1H, H-6b′), 2.59 (bs, 1H, OH), 2.01-1.84 (m, 6H, CH₂), 1.48-1.40 (m, 2H, CH₂), 1.32-1.25 (m, 62H, CH₂), 0.88 (t, J=6.8 Hz, 6H, CH₃×2); ¹³C NMR (150 MHz, CDCl₃) δ 173.1 (C), 138.6 (C), 138.39 (C), 138.35 (C), 138.2 (C×2), 136.7 (CH), 128.4 (CH×2), 128.3 (CH×10), 128.0 (CH×2), 127.91 (CH×2), 127.86 (CH×2), 127.8 (CH), 127.7 (CH), 127.62 (CH), 127.57 (CH), 127.5 (CH), 127.4 (CH×2), 126.5 (CH), 100.0 (CH), 81.3 (CH), 79.2 (CH), 76.6 (CH), 74.8 (CH), 74.5 (CH₂), 74.2 (CH), 73.5 (CH₂), 73.4 (CH₂), 73.1 (CH₂), 71.1 (CH), 69.7 (CH₂), 69.5 (CH₂), 62.3 (CH₂), 50.8 (CH), 36.8 (CH₂), 31.9 (CH₂×2), 29.7 (CH₂×17), 29.63 (CH₂×3), 29.56 (CH₂×3), 29.42 (CH₂), 29.41 (CH₂), 29.3 (CH₂×3), 28.0 (CH₂), 25.7 (CH₂), 22.7 (CH₂×2), 14.1 (CH₃×2); HRMS (ESI, M+H⁺) calculated for C₈₅H₁₂₈O₉N 1306.9584, found 1306.9567.

### Example 25

### Preparation of Compound 17 (2S,3S,4R)-1-O-(2,3,4-tri-O-benzyl-6-O-sulfo-α-D-galacto-pyranosyl)-3,4-di-O-benzyl-2-hexacosanoylamino-5-octade-cen-1,3,4-triol, sodium salt)

To a solution of Compound 16 (245 mg, 0.19 mmol) and SO₃/TMA (130 mg, 0.94 mmol) in DMF (4.0 mL). The reaction flask was warmed up to 50° C., and the mixture was kept stirring for 12 h. After sodium bicarbonate (236 mg, 2.81 mmol) and water (7.5 mL) were added to the solution and stirred for 30 minutes, filtered Compound 17 (258 mg, quant.) was obtained. R_{f} 0.36 (EtOAc); [α]²⁵D −4.88 (c 0.9, CHCl₃); mp 70° C.; IR (CHCl₃) v 3422, 2923, 2853, 1653, 1455, 1149 cm⁻¹; ¹H NMR (600 MHz, CDCl₃) δ 7.35-7.18 (m, 25H, ArH), 6.07 (d, J=8.4 Hz, 1H, NH), 5.71-5.67 (m, 1H, H-6), 5.42 (t, J=10.2 Hz, 1H, H-5), 4.86 (d, J=10.8 Hz, 1H, PhCH₂), 4.80 (d, J=3.6 Hz, 1H, H-1′), 4.74 (d, J=12.0 Hz, 1H, PhCH₂), 4.70-4.61 (m, 6H, PhCH₂), 4.39 (d, J=12.0 Hz, 1H, PhCH₂), 4.33-4.30 (m, 1H, H-2), 4.27 (d, J=12.0 Hz, 1H, PhCH₂), 4.21 (d, J=6.0 Hz, 2H, H-6a′, H-6b′), 4.07-4.04 (m, 3H, H-4, H-4′, H-5′), 3.99 (dd, J=10.2, 3.6 Hz, 1H, H-2′), 3.85 (dd, J=10.2, 2.4 Hz, 1H, H-3′), 3.77-3.72 (m, 2H, H-1a, H-3), 3.62 (dd, J=10.2, 3.0 Hz, 1H, H-1b), 2.05-1.76 (m, 6H, CH₂), 1.40-1.38 (m, 2H, CH₂), 1.31-1.15 (m, 62H, CH₂), 0.88 (t, J=7.2 Hz, 6H, CH₃×2);13C NMR (150 MHz, CDCl₃) δ 174.3 (C), 138.6 (C), 138.4 (C×2), 138.3 (C), 137.5 (C), 137.2 (CH), 128.6 (CH×2), 128.4 (CH×2), 128.28 (CH×4), 128.25 (CH×4), 128.2 (CH×2), 127.9 (CH×2), 127.8 (CH), 127.63 (CH×2), 127.57 (CH), 127.5 (CH×2), 127.4 (CH×3), 126.5 (CH), 98.7 (CH), 80.4 (CH), 78.8 (CH), 76.0 (CH), 74.87 (CH₂), 74.84 (CH), 74.5 (CH₂), 73.5 (CH₂), 73.2 (CH), 72.4 (CH₂), 69.38 (CH), 69.35 (CH₂), 67.0 (CH₂), 66.2 (CH₂), 50.8 (CH), 36.8 (CH₂), 31.9 (CH₂×2), 29.8 (CH₂×8), 29.7 (CH₂×12), 29.6 (CH₂), 29.5 (CH₂×2), 29.40 (CH₂), 29.38 (CH₂×2), 29.35 (CH₂×2), 28.1 (CH₂), 25.9 (CH₂), 22.7 (CH₂×2), 14.1 (CH₃×2); HRMS (ESI, M+Na⁺) calculated for C₈₅H₁₂₆O₁₂NNa₂S 1430.8791, found 1430.8770.

### Example 26

### Preparation of Compound 2h (2S,3S,4R)-1-O-(6-O-sulfo-α-D-galactopyranosyl)-D-ribo-2-hexacosanoylamino-1,3,4-octadecantriol, sodium salt)

Compound 17 (38.4 mg) was dissolved in a mixed solvent of H₂O/MeOH/CHCl₃ (6/3/1 ratio, 1 mL) at room temperature. Pd(OH)₂/C (58.0 mg, Degussa type) was added to the solution, the reaction vessel was purged with hydrogen, and the mixture was stirred under 60 psi pressure at the same temperature for 1 d. The resulting solution was filtered through celite, then saturated sodium bicarbonate (3.0 mL) was added to stir at room temperature for 0.5 h, filtered, and washed the solid to afford the crude Compound 2h (17.1 mg, 65%) as white solid. [α]²⁴_{D} +200.5 (c 0.2, CHCl₃); IR (KBr) v 3350, 2923, 2853, 1639, 1542, 1455, 1257, 1056 cm⁻¹; ¹H NMR (600 MHz, CDCl₃) δ 8.95 (d, J=8.4 Hz, 1H, NH), 5.44 (d, J=3.6 Hz, 1H, H-1′), 5.17-5.13 (m, 1H, H-2), 5.04-4.97 (m, 2H, H-6a′, H-6b′), 4.76 (t, J=6.0 Hz, 1H, H-5′), 4.64-4.58 (m, 2H, H-1a, H-2′), 4.49-4.39 (m, 3H, H-3, H-3′, H-4′), 4.34-4.29 (m, 2H, H-1b, H-4), 2.62-2.56 (m, 2H, CH₂), 2.20-2.15 (m, 1H, H-5a), 1.89-1.73 (m, 3H, H-5b, CH₂), 1.64-1.59 (m, 2H, CH₂), 1.36-1.17 (m, 66H, CH₂), 0.88 (m, 6H, CH₃×2); ¹³C NMR (150 MHz, CDCl₃) δ 174.3 (C), 100.8 (CH), 75.9 (CH), 72.4 (CH), 71.0 (CH), 70.55 (CH), 70.52 (CH), 69.9 (CH), 68.0 (CH₂), 67.6 (CH₂), 51.5 (CH), 36.8 (CH₂), 33.9 (CH₂), 32.07 (CH₂×2), 32.05 (CH₂×2), 30.4 (CH₂), 30.1 (CH₂), 30.0 (CH₂×16), 29.7 (CH₂), 29.59 (CH₂×2), 29.56 (CH₂×2), 26.4 (CH₂×2), 22.9 (CH₂×4), 14.3 (CH₃×2); HRMS (ESI, M+Na⁺) calculated for C₅₀H₉₈O₁₂NNa₂S 982.6600 found 982.6610.

### Example 27

### Preparation of Compound 18 ((2S,3S,4R)-1-O-(2,3,4-Tri-O-benzyl-6-azido-α-D-galactopy-ranosyl)-2-hexacosanoylamino-3,4-O-isopropylidene-5-octa-decen-1,3,4-triol)

To a solution of Compound 9 (98 mg, 0.08 mmol) and triphenylphosphine (66 mg, 0.25 mmol) in tetrahydrofurane (1 mL) at 0° C. was added diisopropylazodicarboxylate (51 μL, 0.25 mmol), followed by the dropwise addition of diphenylphosphorylazide (63 μL, 0.29 mmol). After completion of addition, the temperature of the reaction mixture was brought to 28° C. and stirred for 1 h. Water (5 mL) was added to quench the reaction and the mixture was extracted with ethyl acetate (3×5 mL). The combined organic layers were washed with brine, dried over anhydrous MgSO₄, filtered, and concentrated in vacuo to give a residue. The residue was purified by column chromatography to give the azide Compound 18 (100 mg, 99%) as white solid. R_{f} 0.71 (EtOAc/Hex=1/2.5); [α]²⁵_{D} +17.0 (c 0.6, CHCl₃); mp 80-82° C.; IR (CHCl₃) v 3309, 2918, 2850, 2095, 1641, 1546, 1469, 1042 cm⁻¹; ¹H NMR (600 MHz, CDCl₃) δ 7.42-7.25 (m, 15H, ArH), 8.89 (d, J=9.0 Hz, 1H, NH), 5.60 (td, J=10.8, 7.2 Hz, 1H, H-6), 5.44 (t, J=9.6 Hz, 1H, H-5), 5.02-4.98 (m, 2H, H-1′, PhCH₂), 4.88 (dd, J=9.6, 6.6 Hz, 1H, H-4), 4.85 (d, J=12.0 Hz, 1H, PhCH₂), 4.81 (d, J=10.8 Hz, 1H, PhCH₂), 4.77 (d, J=11.4 Hz, 1H, PhCH₂), 4.69 (d, J=11.4 Hz, 1H, PhCH₂), 4.60 (d, J=12.0 Hz, 1H, PhCH₂), 4.18 (dd, J=7.8, 5.4 Hz, 1H, H-3), 4.14-4.10 (m, 1H, H-2), 4.05 (dd, J=10.2, 3.6 Hz, 1H, H-2′), 3.91 (dd, J=12.0, 2.4 Hz, 1H, H-3′), 3.89 (dd, J=11.4, 3.6 Hz, 1H, H-1a), 3.83-3.81 (m, 2H, H-4′, H-5′), 3.69 (dd, J=11.4, 7.8 Hz, 1H, H-1b), 3.52 (dd, J=12.0, 7.8 Hz, 1H, H-6a′), 3.04 (dd, J=12.0, 4.8 Hz, 1H, H-6b′), 2.11-1.90 (m, 2H, CH₂), 1.56-1.51 (m, 2H, CH₂), 1.46 (s, 3H, CH₃), 1.36 (s, 3H, CH₃), 1.25 (bs, 64H, CH₂), 0.88 (t, J=7.2 Hz, 6H, CH₃×2); ¹³C NMR (150 MHz, CDCl₃) δ 172.3 (C), 138.4 (C), 138.2 (C), 138.0 (C), 135.1 (CH), 130.0 (CH×3), 128.4 (CH×3), 127.94 (CH), 127.89 (CH), 127.87 (CH), 127.7 (CH), 127.5 (CH), 126.1 (CH×2), 124.0 (CH), 120.22 (CH), 120.18 (CH), 108.4 (C), 98.8 (CH), 78.7 (CH), 76.6 (CH), 76.3 (CH), 74.65 (CH₂), 74.63 (CH), 73.4 (CH₂), 73.12 (CH₂), 73.06 (CH), 69.8 (CH), 68.9 (CH₂), 51.4 (CH₂), 49.0 (CH), 36.8 (CH₂), 31.9 (CH₂), 29.7 (CH₂×24), 29.6 (CH₂), 29.5 (CH₂), 29.45 (CH₂), 29.42 (CH₂), 29.3 (CH₂×2), 27.8 (CH₃), 27.7 (CH₂), 25.6 (CH₂), 25.5 (CH₃), 22.7 (CH₂), 14.1 (CH₃×2); HRMS (ESI, M+H⁺) calculated for C₇₄H₁₁₉O₈N₄ 1191.9022, found 1191.9016.

### Example 28

### Preparation of Compound 2i (2S,3S,4R)-1-O-(6-amine-α-D-galactopyranosyl)-D-ribo-2-hexacosanoylamino-1,3,4-octadecantriol)

Compound 18 (73 mg) was dissolved in a mixed solvent of MeOH/CHCl₃ (3/1 ratio, 4 mL) at 28° C. Pd(OH)₂/C (73 mg, Degussa type) was added to the solution and added 2-3 drop acetic acid, the reaction vessel was purged with hydrogen, and the mixture was stirred under 60 psi pressure at the same temperature for 5 h. The resulting solution was filter through celite, the filtrate was concentrated in vacuo, and the residue was purified by column chromatography to afford Compound 2i (17 mg, 31%) as white solid. R_{f} 0.2 (MeOH/DCM=1/4); the poor solubility of this amine compound at room temperature prevented us from obtaining reliable optical rotation data. Mp 187-188° C.; IR (KBr) v 3417, 2920, 2851, 1645, 1072 cm⁻¹; ¹H NMR (600 MHz, d-pyridine, 100° C.) δ 8.02 (bs, 1H, NH), 5.35 (d, J=2.4 Hz, 1H, H-1′), 5.02 (bs, 1H, H-2), 4.87 (d, J=3.0 Hz, 1H, H-5′), 4.64 (dd, J=10.2, 4.8 Hz, 1H, H-1a), 4.39 (dd, J=9.0, 3.6 Hz, 1H, H-2′), 4.34-4.33 (m, 2H, H-3′, H-4′), 4.19-4.16 (m, 3H, H-1b, H-3, H-4), 3.85 (dd, J=13.2, 7.8 Hz, 1H, H-6a′), 3.65 (dd, J=12.6, 2.4 Hz, 1H, H-6b′), 2.46 (t, J=7.2 Hz, 2H, CH₂), 2.40 (t, J=7.8 Hz, 1H, CH₂), 2.20-2.15 (m, 1H, CH₂), 1.84-1.83(m, 4H, CH₂), 1.75-1.65 (m, 3H, CH₂), 1.40 (bs, 34H, CH₂), 1.35 (bs, 29H, CH₂), 0.93 (t, J=6.6 Hz, 6H, CH₃×2); ¹³C NMR (150 MHz, d-pyridine, 100° C.) δ 174.0 (C), 101.8 (CH), 77.3 (CH), 73.0 (CH), 71.6 (CH), 71.2 (CH), 70.2 (CH), 69.8 (CH₂), 68.5 (CH), 52.9 (CH), 42.0 (CH₂), 37.2 (CH₂), 34.84 (CH₂), 34.78 (CH₂), 34.6 (CH₂), 32.3 (CH₂×3), 31.2 (CH₂), 30.6 (CH₂), 30.5 (CH₂×2), 30.2 (CH₂×2), 30.1 (CH₂×7), 29.94 (CH₂×3), 29.91 (CH₂×2), 29.7 (CH₂×3), 29.52 (CH₂), 29.46 (CH₂), 27.4 (CH₂), 26.5 (CH₂×2), 24.6 (CH₂), 23.0 (CH₂×3), 14.2 (CH₃×2); HRMS (ESI, M+H⁺) calculated for C₅₀H₁₀₁O₈N₂ 857.7552, found 857.7558.

### Example 29

### In Vitro Evaluation of the Immune Response Elicited by Compounds 1 and 2a-2i

The immune response elicited by Compounds 1 and 2a-2i was assessed by the induction of IL-2 in mNK1.2 cells. Compound 1 is illustrated by the following formula:

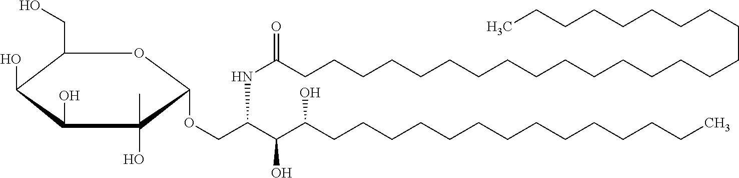

Method: A20-CD 1d cells were loaded with Compound 1 (α-GalCer) and Compounds 2a-2i, and cultured with mNK1.2 cells. Supernatants were collected after 72 hours to determine the production of IL-2 by ELISA.

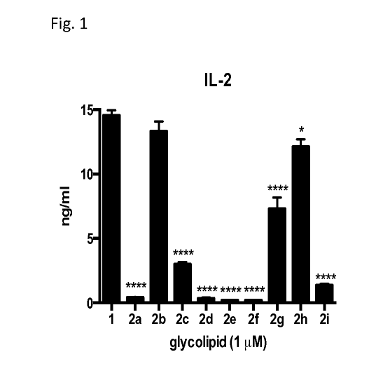

Results: As shown in FIG. 1, the IL-2 levels induced by Compound 1 (14.5±0.6 ng/mL) and compound 2b (13.3±1.3 ng/mL) were significantly higher than those other Compounds (range: 0.17±0.07-12.12±1.0 ng/mL, p<0.05). Without being bound by any particular theory, these findings suggest that longer acyl chain at Gal 6′ of α-GalCer may diminish the activation of murine NKT cell.

The immune response elicited by Compounds 1 and 2a-2i in human NKT cells was evaluated using human dendritic cell (DC).

Method: Human iNKT cells were isolated with anti-TCR Vα24 antibody and cultured for 7 days in the presence of IL-2 (1 μg/mL). The dendritic cells were generated from CD14+ cells, sorted from peripheral blood mononuclear cells (PBMC) by incubating PBMC for 7 days with GM-CSF (50 ng/mL) and IL-4 (50 ng/mL). Dendritic cells were loaded with the following: 1 μM of Compound 1 or 1 μM of Compounds 2a-2i, and cultured with iNKT cells for 3 days. The culture supernatants were collected and analyzed for various cytokines production by Luminex. Data were presented as mean±SD and analyzed by one-way ANOVA.

Results: Compounds 2b (2286±344.3 pg/mL), 2g (2704±10.3 pg/mL), 2h (2739±14.52 pg/mL) and 2i (2687±89.4 pg/mL) induced comparable levels of IFN-γ (an Th1 cytokine) as Compound 1 (2493±302.6 pg/mL).

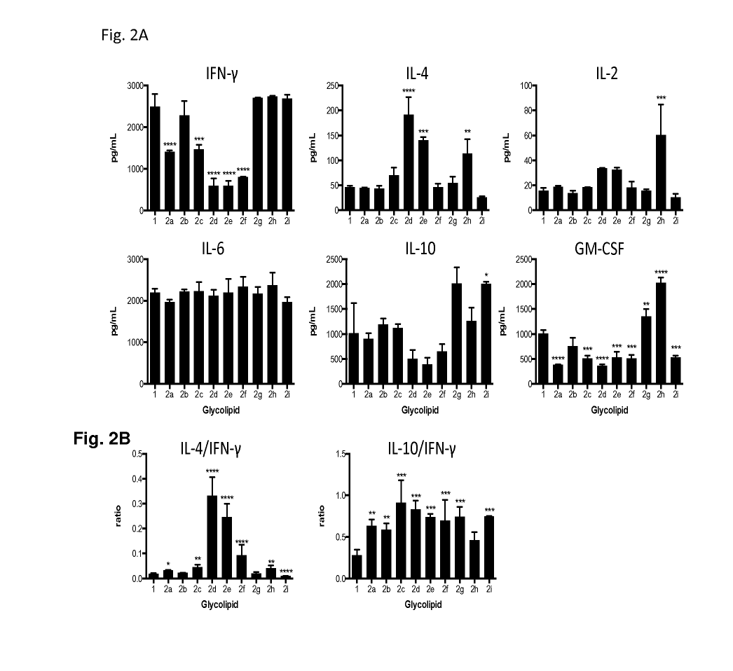

Compound 2d (33.8±0.2 pg/mL), 2e (32.5±1.7 pg/mL) and 2h (60.3±24.4 pg/mL) were more effective in IL-2 (an Th1 cytokine) induction than Compound 1 (15.6±2.3 pg/mL) (see FIG. 2A).

Compounds 2d (191.5±35.3 pg/mL, p<0.0001), 2e (140.4±6.1 pg/mL, p<0.001) and 2h (113.9±28.4 pg/mL, p<0.01) were significantly more effective than Compound 1 (46.3±2.8 pg/mL) in IL-4 induction (an Th2 cytokine, see FIG. 2A).

Compounds 2g (2010±325.1 pg/mL) and 2i (2001±46.8 pg/mL) effectively increased the level of IL-10 (an Th2 cytokine) compare to Compound 1 (1017±603.4 pg/mL, p<0.05) (FIG. 2A).

The induction of IL-6 was comparable between Compound 1 (2192±92.9 pg/mL) and Compounds 2a-2i (range from 1963±120.9 to 2368±308.7 pg/mL).

The induction of GM-CSF by compound 2g (1350±146.2 pg/mL, p<0.01) and 2h (2024±108.4 pg/mL, p<0.0001) was significantly higher than that of Compound 1 (1011±67.1 pg/mL). Previous study by C. H. Wong et al (Bioorg. Med. Chem Lett, 2005) has reported that modification of 3′-OH of galactose moiety with a sulfate group (SO4Na2) induced comparable levels of IFN-γ and IL-4 as Compound 1. The data shows modification of 6′-OH of galactose moiety with a sulfate group elicited comparable level of IFN-γ, but a higher level of IL-4, IL-2 and GM-CSF than Compound 1. Therefore, the modification at 6′-OH of galactose with a sulfate group is better than the modification at 3′-OH of galatose in stimulating NKT cells and immune response.

The ratios of IL-4/IFN-γ and IL-10/IFN-γ were used evaluate if the immune response elicited by Compounds 1 and 2a-2i was skewed toward Th2 mediated response.

The ratios of IL-4/IFN-γ and IL-10/IFN-γ (FIG. 2B) were significantly higher for compound 2a (0.032±0.0009 and 0.63±0.07), 2c (0.044±0.011 and 0.91±0.26), 2d (0.331±0.074 and 0.83±0.1), 2e (0.246±0.053 and 0.73±0.03) and 2f (0.093±0.041 and 0.69±0.24) compared to Compound 1 (0.018±0.003 and 0.28±0.06). Without being bound by any particular theory, it is believed that an acyl chain with 12-13 carbon atoms at 6′-OH of galactose moiety triggers a stronger Th2 immune response.

The levels of cytokines induced by Compound 2b were similar to those of Compound 1. Without being bound by any particular theory, it is believed that modification of 6′-OH of galactose moiety with a methyl group does not change its ability to activate NKT cells compare to Compound 1.

The production of IFN-γ was decreased and production of IL-4 was increased when the number of carbon atoms of the acyl chain increased from 6 to 13.

The ratios of IL-4/IFN-γ and IL-10/IFN-γ for a well-known Th2-biased glycolipid, (OCH), were 0.25 and 0.26. These results indicate that compound 2d and 2e may skew the immune responses toward Th2 response more potently than Compound 1 and at least equal to or better than that of OCH.

Compound 2i showed a comparable level of IFN-γ, lower level of IL-4 and lower ratio of IL-4/IFN-γ compared to Compound 1, indicating that Compound 2i is more potent in inducing Th1 mediated immune response.

What is claimed is:

1. A compound of formula (I) 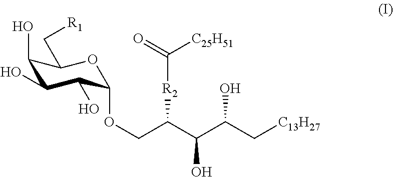
or a pharmaceutically acceptable salt thereof,
R₁ is one of —O—R₃, —R₇N(R₈)R₉, —R₇₁N⁺(R₇₂R₇₃R₇₄)X⁻, —R₁₁C(═NR₁₂)R₁₃, —R₁₄—N₃, —R₁₅—N═N—R₁₆, or —R₁₇(C═O)R₁₈(C═O)R₁₉, —R₂₀N(COR₂₁)(COR₂₂), where R₇ is a bond or alkenyl; each of R₈ and R₉ is independently a hydrogen, an alkyl or an alkenyl; R₇₁ is a bond or alkenyl; each of R₇₂, R₇₃ and R₇₄ is independently an alkyl or an alkenyl; X is a halogen; R₁₁ is a bond or an alkenyl; R₁₂ is an alkyl or alkenyl; R₁₃ is an alkyl or an alkenyl; R₁₄ is a bond or an alkenyl; R₁₅ is a bond or an alkenyl; R₁₆ is an alkyl or alkenyl; R₁₇ is a bond or alkenyl; R₁₈ is an alkenyl; R₁₉ is an alkyl or alkenyl; R₂₀ is a bond or an alkenyl; R₂₁ is an alkyl or an alkenyl; R₂₂ is an alkyl or an alkenyl;
R₂ is N—R₅ where R₅ is hydrogen or an alkyl;
R₃ is alkyl, alkenyl, —PO₃H₂, —SO₃Na, —SO₃K, —SO₃Li, —SH, —SR₆, —SSR₆, —SOR₆, —SO₂R₆, —SO₂H, —SO₃H, —SO₃R₆, —SCN, —R₆P, —OP(═O)(OH)₂ or —OPO(OR₆)₂, where R₆ is alkyl or an alkenyl;
provided R₁ is not OCH₃ or NH₂ when R₂ is NH.

2. The compound of claim 1, wherein said R₃ is C₂H₅ alkyl to C₃₀H₆₁alkyl.

3. The compound of claim 1, wherein R₅ is CH₃ to C₆H₁₃.

4. The compound of claim 1, wherein R₃ is CH₂ and R₂ is NCH₃

5. The compound of claim 1, wherein R₃ is C₆H₁₃-C₂₀H₄₁, PO₃H₂ or SO₃Na and R₂ is NH.

6. A pharmaceutical composition comprising a compound of formula (I) 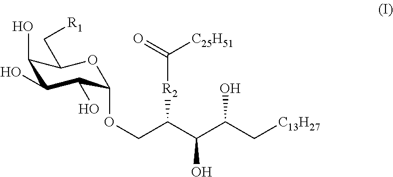
or a pharmaceutically acceptable salt thereof,
R₁ is one of —O—R₃, —R₇N(R₈)R₉, —R₇₁N⁺(R₇₂R₇₃R₇₄)X⁻, —R₁₁C(═NR₁₂)R₁₃, —R₁₄—N₃, —R₁₅—N═N—R₁₆, or —R₁₇(C═O)R₁₈(C═O)R₁₉, —R₂₀N(COR₂₁)(COR₂₂), where R₇ is a bond or alkenyl; each of R₈ and R₉ is independently a hydrogen, an alkyl or an alkenyl; R₇₁ is a bond or alkenyl; each of R₇₂, R₇₃ and R₇₄ is independently an alkyl or an alkenyl; X is a halogen; R₁₁ is a bond or an alkenyl; R₁₂ is an alkyl or alkenyl; R₁₃ is an alkyl or an alkenyl; R₁₄ is a bond or an alkenyl; R₁₅ is a bond or an alkenyl; R₁₆ is an alkyl or alkenyl; R₁₇ is a bond or alkenyl; R₁₈ is an alkenyl; R₁₉ is an alkyl or alkenyl; R₂₀ is a bond or an alkenyl; R₂₁ is an alkyl or an alkenyl; R₂₂ is an alkyl or an alkenyl;
R₂ is N—R₅ where R₅ is hydrogen or an alkyl;
R₃ is alkyl, alkenyl, PO₃H₂, —SO₃Na, —SO₃K, SO₃Li, —SH, —SR₆, —SSR₆, —SOR₆, —SO₂R₆, —SO₂H, —SO₃H, —SO₃R₆, —SCN, —R₆P, —OP(═O)(OH)₂ or —OPO(OR₆)₂, where R6 is alkyl or an alkenyl;
provided R₁ is not OCH₃ or NH₂ when R₂ is NH, and
a pharmaceutically acceptable carrier.

7. The pharmaceutical composition of claim 6, wherein said R₃ is C₂H₅ alkyl to C₃₀H₆₁alkyl.

8. The pharmaceutical composition of claim 6, wherein R₅ is CH₃ to C₆H₁₃.

9. The pharmaceutical composition of claim 6, wherein R₃ is CH₂ and R₂ is NCH₃

10. The pharmaceutical composition of claim 6, wherein R₃ is C₆H₁₃-C₂₀H₄₁, PO₃H₂ or SO₃Na and R₂ is NH.

11. A method of preparing compounds of formula (2a) 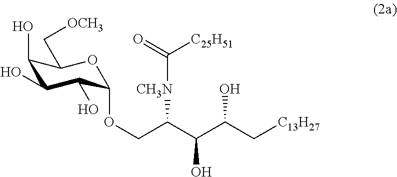
comprising:
(a) providing a compound having formula (9) 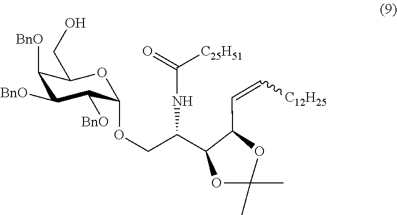
(b) reacting the compound in (a) with
i) a base; and
ii) methyl iodide or DMS, to produce a compound having the formula (10a) 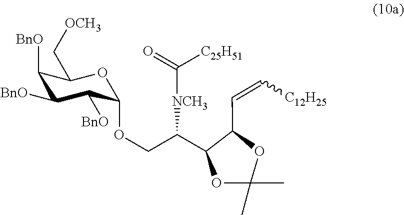
(c) converting the compound obtained in (b) to the compound having formula 2a.

12. The method of claim 11, wherein the reaction in (c) comprises reacting the compound obtained in (b) with a mixture containing palladium hydroxide, methanol, acetic acid, chloroform, and hydrogen gas.

13. The method of claim 11, wherein the reaction in (b) is carried out in DMF or ether at about 0° C. to about 35° C.

14. The method of claim 13, wherein ether is THF or 1,4-dioxane.

15. The method of claim 11, wherein the base in reaction (b) is NaH.

16. The method of claim 15, wherein for the reaction in (b), each of base NaH and methyl iodide is added in an amount at a molar ratio of 2:1 of the amount of compound (9) obtained in (a).

17. A method of preparing compounds having formula (2g) 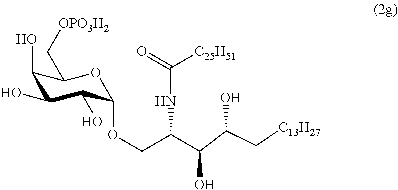
comprising:
(a) providing a compound having formula (9) 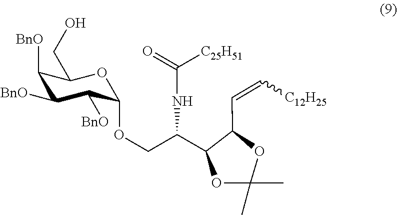
(b) reacting the compound (9) in (a) with DPPA to produce a compound having formula (11g) 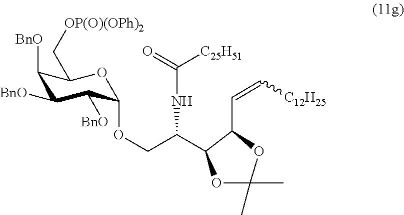
(c) hydrolyzing the acetonide group of the compound 11g obtained from (b) to produce a diol compound having formula 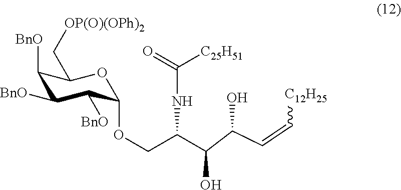
(d) converting the compound (12) obtained in (c) to the compound having formula 2g.

18. The method of claim 17, wherein the reaction in (b) is carried out at about 4° C. to −10° C. in the presence of a base in dichloromethane.

19. The method of claim 18, wherein the base is 1,8-diazabicyclo[5.4.0]undec-7-ene.

20. The method of claim 17, wherein hydrolyzing the acetonide group of the compound obtained from (b) comprises reacting the compound obtained from (b) with an acid in ether.

21. The method of claim 20, wherein the acid is sulfuric acid and ether is 1,4-dioxane.

22. The method of claim 17, wherein the reaction in (d) comprises:
(i) reacting the compound obtained in (c) with a mixture containing palladium hydroxide, methanol, chloroform, and hydrogen gas, thereby obtaining a solution;
(ii) concentrating the solution in (i) to obtain a residue;
(iii) dissolving the residue in (ii) with a mixture of methanol and chloroform to obtain a second solution;
(iv) adding PtO₂ to the second solution; and
(v) passing hydrogen gas through the second solution.

23. A method of preparing compounds having formula (2h): 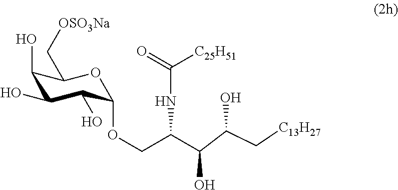
comprising:
(a) providing a compound having formula 8 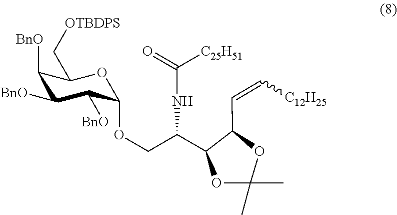
(b) hydrolyzing the acetonide group in the compound (8) obtained from (a) to produce a diol compound having the formula (14) 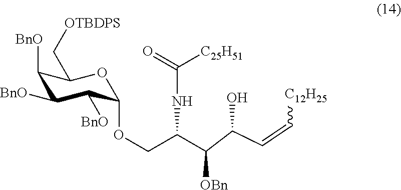
(c) benzylating the diol compound (14) produced in (b) to form a compound having the formula (15) 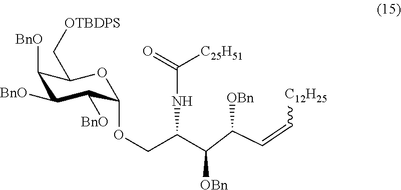
(d) hydrolyzing the compound (15) obtained in (c) to produce a compound having the formula (16) 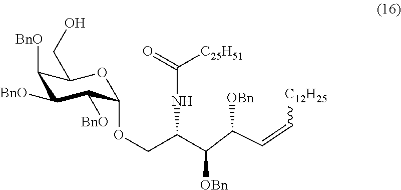
(e) converting the compound (16) obtained in (d) to the compound having formula (17). 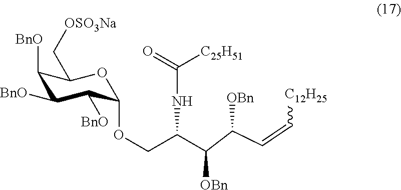
(f) converting the compound (17) obtained in (e) to the compound having the formula (2h).

24. The method of claim 23, wherein hydrolyzing the acetonide group in the compound obtained from (a) is by an acid in ether.

25. The method of claim 24, wherein the acid is sulfuric acid and the ether is 1,4-dioxane.

26. The method of claim 23, wherein benzylation of the compound obtained from (b) is by benzyl bromide in the presence of NaH in tetrahydrofurane.

27. The method of claim 23, wherein hydrolyzing a TBDPS group in the compound (15) from (c) is by TBAF in THF.

28. The method of claim 23, wherein converting the compound obtained in (d) to the compound having formula (17) in (e) comprises reacting the compound obtained in (d) with sulfur trioxide trimethylamine complex.

29. The method of claim 23, wherein converting the compound obtained in (e) to the compound having formula (2h) comprises reacting with a mixture containing palladium hydroxide, methanol, chloroform, and hydrogen gas.

30. A method of preparing compounds having formula (2i) 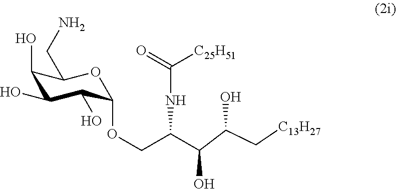
comprising:
(a) providing a compound having the formula (9); 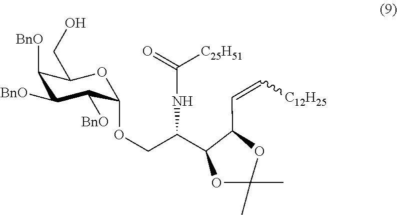
(b) converting the compound (9) in (a) to an azide compound having the formula (18) 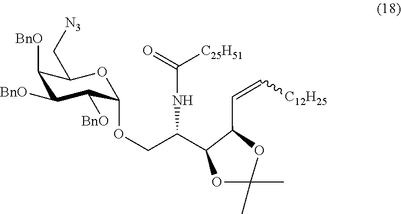
(c) converting the compound (18) obtained in (b) to form compound having the formula 2i.

31. The method of claim 30, wherein the reaction in (b) comprises:
(i) adding the compound in (a) to tetrahydrofurane containing triphenylphosphine to form a solution;
(ii) adding diisopropylazodicarboxylate and diphenylphosphorylazide to the solution in (i);
(iii) concentrating the solution in (ii) and isolating the azide compound having formula (18).

32. The method of claim 30, wherein the reaction in (c) comprises reacting the azide compound obtained in (b) with a mixture containing palladium hydroxide, methanol, acetic acid, chloroform, and hydrogen gas.
